(19) United States
(12) Patent Application Publication
(10) Pub. No.: US 2010/0009967 A1
(43) Pub. Date: Jan. 14, 2010

(54) SOLID DOSAGE FORMULATIONS OF TELCAGEPANT POTASSIUM

(76) Inventors: Majid Mahjour, Schwenksville, PA (US); Dina Zhang, Watchung, NJ (US); Aaron J. Moment, Edison, NJ (US)

Correspondence Address: MERCK AND CO., INC, P O BOX 2000, RAHWAY, NJ, 07065-0907, US

(21) Appl. No.: 12/493,311

(22) Filed: Jun. 29, 2009

Related U.S. Application Data
(60) Provisional application No. 61/133,542, filed on Jun. 30, 2008.

(51) Int. Cl. A61K 31/55; C07D 401/14

(52) U.S. Cl. 514/212.08; 540/524

(57) ABSTRACT

A solid dosage pharmaceutical formulation comprising as an active ingredient the potassium salt of N-[(3R,6S)-6-(2,3-difluorophenyl)-2-oxo-1-(2,2,2-trifluoroethyl)azepan-3-yl]-4-(2-oxo-2,3-dihydro-1H-imidazo[4,5-b]pyridin-1-yl)piperidine-1-carboxamide (telcagepant), arginine, and a pharmaceutically acceptable surfactant. The invention is also directed to an amorphous form of the potassium salt of telcagepant.

### CROSS-REFERENCE TO RELATED APPLICATIONS

This application claims benefit under 35 U.S.C. §119(e) of U.S. provisional application Ser. No. 61/133,542, filed Jun. 30, 2008.

### FIELD OF THE INVENTION

The field of the invention is solid dosage pharmaceutical formulations. More specifically, the field of the invention is the formulation of active ingredients in oral solid dosage forms.

### BACKGROUND OF THE INVENTION

CGRP (Calcitonin Gene-Related Peptide) is a naturally occurring 37-amino acid peptide that is generated by tissue-specific alternate processing of calcitonin messenger RNA and is widely distributed in the central and peripheral nervous system. CGRP is localized predominantly in sensory afferent and central neurons and mediates several biological actions, including vasodilation. When released from the cell, CGRP initiates its biological responses by binding to specific cell surface receptors that are predominantly coupled to the activation of adenylyl cyclase. CGRP receptors have been identified and pharmacologically evaluated in several tissues and cells, including those of brain, cardiovascular, endothelial, and smooth muscle origin.

CGRP is a potent neuromodulator that has been implicated in the pathology of cerebrovascular disorders such as migraine and cluster headache. In clinical studies, elevated levels of CGRP in the jugular vein were found to occur during migraine attacks (Goadsby et al., Ann. Neurol., 1990, 28, 183-187), and salivary levels of CGRP were shown to be elevated in migraine subjects between attacks (Bellamy et al., Headache, 2006, 46, 24-33). CGRP itself has been shown to trigger migrainous headache (Lassen et al., Cephalalgia, 2002, 22, 54-61). In clinical trials, the CGRP antagonist BIBN4096BS has been shown to be effective in treating acute attacks of migraine (Olesen et al., New Engl. J. Med., 2004, 350, 1104-1110) and was able to prevent headache induced by CGRP infusion in a control group (Petersen et al., Clin. Pharmacol. Ther., 2005, 77, 202-213).

CGRP-mediated activation of the trigeminovascular system may play a key role in migraine pathogenesis. Additionally, CGRP activates receptors on the smooth muscle of intracranial vessels, leading to increased vasodilation, which is thought to contribute to headache pain during migraine attacks (Lance, Headache Pathogenesis: Monoamines, Neuropeptides, Purines and Nitric Oxide, Lippincott-Raven Publishers, 1997, 3-9). The middle meningeal artery, the principle artery in the dura mater, is innervated by sensory fibers from the trigeminal ganglion which contain several neuropeptides, including CGRP. Trigeminal ganglion stimulation in the cat resulted in increased levels of CGRP, and in humans, activation of the trigeminal system caused facial flushing and increased levels of CGRP in the external jugular vein (Goadsby et al., Ann. Neurol., 1988, 23, 193-196). Thus the vascular effects of CGRP may be attenuated, prevented or reversed by a CGRP antagonist.

CGRP antagonist compounds are useful as pharmacological agents for disorders that involve CGRP in humans and animals, but particularly in humans. In addition to headaches, such disorders include pain; non-insulin dependent diabetes mellitus; vascular disorders; inflammation; arthritis; bronchial hyperreactivity; asthma; shock; sepsis; opiate withdrawal syndrome; morphine tolerance; hot flashes in men and women; allergic dermatitis; psoriasis; encephalitis; brain trauma; ischaemia; stroke; epilepsy; neurodegenerative diseases; skin diseases; neurogenic cutaneous redness, skin rosaceousness and erythema; tinnitus; inflammatory bowel disease; irritable bowel syndrome; and cystitis. Of particular importance is the acute or prophylactic treatment of headache, including migraine and cluster headache.

International patent application W02004/092166, published Oct. 28, 2004, discloses compounds useful for the treatment of diseases or conditions of humans or other species which can be treated with inhibitors, modulators or promoters of the CGRP receptor function. Such diseases or conditions include those mentioned in the referenced applications, and specifically include migraine and cluster headache.

Example 86 of WO '166, N-[(3R,6S)-6-(2,3-Difluorophenyl)-2-oxo-1-(2,2,2-trifluoroethyl)azepan-3-yl]-4-(2-oxo-2,3-dihydro-1H-imidazo[4,5-b]pyridin-1-yl)piperidine-1-carboxamide (telcagepant or compound 1):

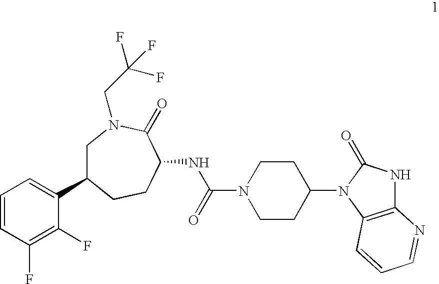

is a particularly potent CGRP modulator. The laboratory preparation of compound 1 is described in WO '166.

International patent application publication WO 2007/120592 discloses the potassium salt of compound 1 (“telcagepant” or compound 1A), the potassium salt hydrate (compound 1B or “telcagepant potassium hydrate”), and the potassium salt ethanolate (compound 1C or “telcagepant potassium ethanolate”):

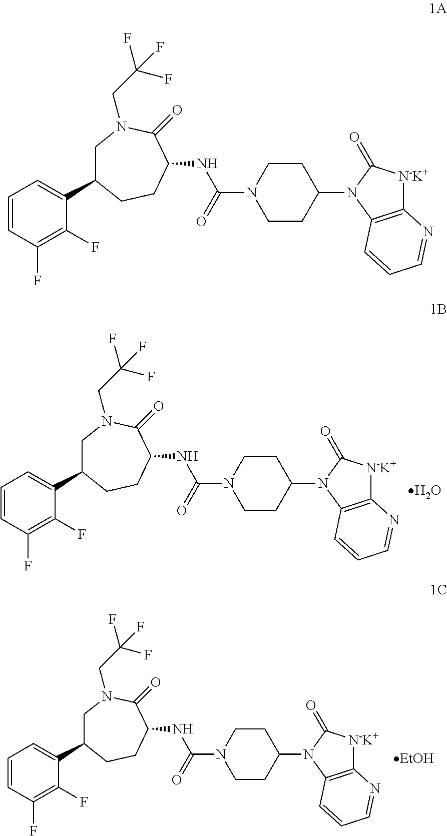

International patent application publication WO 2008/030524 describes liquid formulations of compound 1, and salts and solvate forms thereof.

Telcagepant is currently in clinical development for the treatment of migraine.

### SUMMARY OF THE INVENTION

The invention is directed to a solid dosage pharmaceutical formulation comprising as an active ingredient the potassium salt of N-[(3R,6S)-6-(2,3-difluorophenyl)-2-oxo-1-(2,2,2-trifluoroethyl)azepan-3-yl]-4-(2-oxo-2,3-dihydro-1H-imidazo[4,5-b]pyridin-1-yl)piperidine-1-carboxamide (telcagepant), arginine and a pharmaceutically acceptable surfactant. In particular embodiments, the active ingredient is the ethanolate or hydrate, or an amorphous form, of telcagepant potassium. When the active ingredient is the ethanolate, the compositions of the invention comprise Form I or Form II, or mixtures thereof, of the telcagepant potassium ethanolate.

The invention is also directed to a novel amorphous form of the potassium salt of telcagepant.

### FIGURES OF THE INVENTION

FIG. 1 is a flow diagram that describes a process for manufacturing a solid dosage formulation of the invention;

FIGS. 2A and 2B are photographs of the powder amorphous form of the potassium salt of telcagepant, manufactured by the spray drying method;

FIGS. 3A and 3B are photographs of the powder amorphous form of the potassium salt of telcagepant, manufactured by the precipitation method;

FIG. 4 is an X-ray diffraction pattern of telcagepant potassium ethanolate Form I;

FIG. 5 is an X-ray diffraction pattern of telcagepant potassium ethanolate Form II;

FIG. 6 is an X-ray diffraction pattern of telcagepant potassium hydrate;

FIG. 7 is a modulated DSC curve of the amorphous form of telcagepant potassium;

FIG. 8 is a carbon-13 cross-polarization magic-angle spinning (CPMAS) nuclear magnetic resonance (NMR) spectrum of the crystalline telcagepant potassium ethanolate (Form I) of telcagepant;

FIG. 9 is a carbon-13 cross-polarization magic-angle spinning (CPMAS) nuclear magnetic resonance (NMR) spectrum of the crystalline telcagepant potassium hydrate;

FIG. 10 is a carbon-13 cross-polarization magic-angle spinning (CPMAS) nuclear magnetic resonance (NMR) spectrum of amorphous telcagepant potassium;

FIG. 11 is a Raman spectrum of telcagepant potassium ethanolate Form I;

FIG. 12 is a Raman spectrum of telcagepant potassium hydrate;

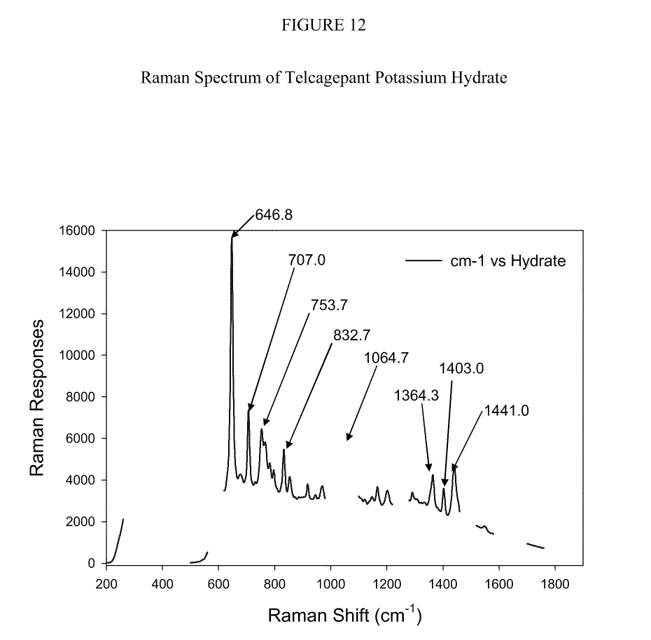

FIG. 13 is a Raman spectrum of the amorphous form of telcagepant potassium;

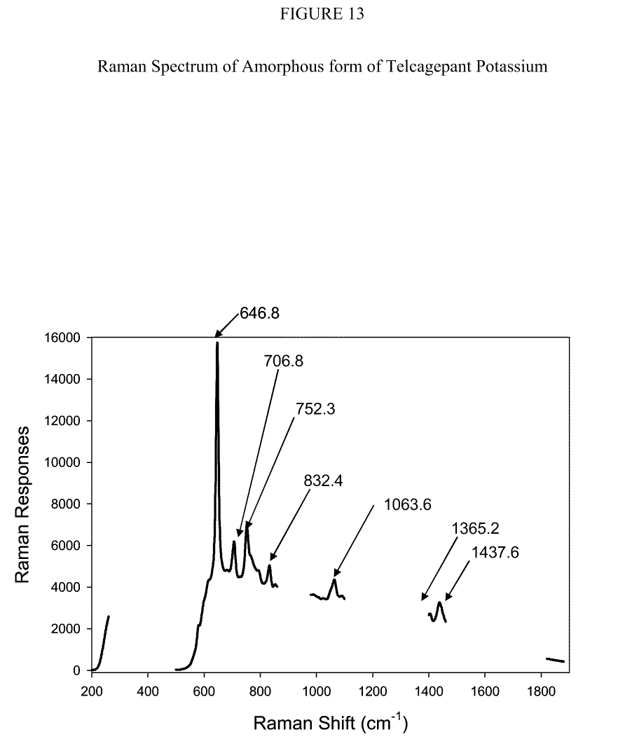

FIG. 14 depicts the preliminary mean plasma concentration-time profile, following administration of a 300 mg single oral dose of telcagepant potassium ethanolate.

### DETAILED DESCRIPTION OF THE INVENTION

The invention is directed to a solid dosage pharmaceutical formulation comprising

(1) N-[(3R,6S)-6-(2,3-difluorophenyl)-2-oxo-1-(2,2,2-trifluoroethyl)azepan-3-yl]-4-(2-oxo-2,3-dihydro-1H-imidazo[4,5-b]pyridin-1-yl)piperidine-1-carboxamide potassium, or the hydrate or ethanolate thereof, or an amorphous form thereof,

(2) arginine; and

(3) a pharmaceutically acceptable surfactant.

In particular embodiments, the formulation comprises the ethanolate of N-[(3R,6S)-6-(2,3-difluorophenyl)-2-oxo-1-(2,2,2-trifluoroethyl)azepan-3-yl]-4-(2-oxo-2,3-dihydro-1H-imidazo[4,5-b]pyridin-1-yl)piperidine-1-carboxamide potassium.

In other embodiments, the formulation comprises Form I or Form II, or mixtures thereof, of the ethanolate of N-[(3R,6S)-6-(2,3-difluorophenyl)-2-oxo-1-(2,2,2-trifluoroethyl)azepan-3-yl]-4-(2-oxo-2,3-dihydro-1H-imidazo[4,5-b]pyridin-1-yl)piperidine-1-carboxamide potassium.

As used in the compositions of the invention, Form I can be detected by one or more of its characteristic x-ray diffraction peaks as described herein, such as d-spacings of 8.27, 4.01, and 3.32 angstroms.

As used in the compositions of the invention, Form I can be detected by one or more of its characteristic solid-state carbon-13 NMR spectra peaks as described herein, such as 109.1 ppm, 55.8 ppm and 54.6 ppm.

As used in the compositions of the invention, Form I can be detected by one or more of its characteristic Raman spectra as described herein, for example at peaks (cm⁻¹) of 646.3, 707.4, 761.5, 832.9, 1063.3, 1365.5, 1402.0, 1445.7 or 1455.3.

As used in the compositions of the invention, Form II can be detected by one or more of its characteristic x-ray diffraction peaks as described herein, such as d-spacings of 11.62, 7.80, and 4.92 angstroms.

In other embodiments, the formulation comprises the hydrate of N-[(3R,6S)-6-(2,3-difluorophenyl)-2-oxo-1-(2,2,2-trifluoroethyl)azepan-3-yl]-4-(2-oxo-2,3-dihydro-1H-imidazo[4,5-b]pyridin-1-yl)piperidine-1-carboxamide potassium.

As used in the compositions of the invention, the hydrate can be detected by one or more of its characteristic x-ray diffraction peaks as described herein, such as d-spacings of 16.96, 8.50, and 4.26 angstroms.

As used in the compositions of the invention, the hydrate can be detected by one or more of its characteristic solid-state carbon-13 NMR spectra peaks as described herein, such as 126.1 ppm, 54.4 ppm and 36.6 ppm.

As used in the compositions of the invention, the hydrate can be detected by one or more of its characteristic Raman spectra as described herein, for example by peaks (cm⁻¹) of 646.8, 707.0, 753.7, 832.7, 1064.7, 1364.3, 1403.0 or 1441.0.

In another embodiment, the formulation comprises an amorphous form of N-[(3R,6S)-6-(2,3-difluorophenyl)-2-oxo-1-(2,2,2-trifluoroethyl)azepan-3-yl]-4-(2-oxo-2,3-dihydro-1H-imidazo[4,5-b]pyridin-1-yl)piperidine-1-carboxamide potassium.

The formulation may comprise from about 0.005 mg to about 1000 mg of the active ingredient N-[(3R,6S)-6-(2,3-difluorophenyl)-2-oxo-1-(2,2,2-trifluoroethyl)azepan-3-yl]-4-(2-oxo-2,3-dihydro-1H-imidazo[4,5-b]pyridin-1-yl)piperidine-1-carboxamide, which is determined from an equivalent weight measurement of N-[(3R,6S)-6-(2,3-difluorophenyl)-2-oxo-1-(2,2,2-trifluoroethyl)azepan-3-yl]-4-(2-oxo-2,3-dihydro-1H-imidazo[4,5-b]pyridin-1-yl)piperidine-1-carboxamide potassium telcagepant, as the hydrate, ethanolate, or amorphous form. Suitable formulations may comprise from 10 to 800 mg, or from 25 to 750 mg, or from 50 to 700, or from 100 to 500 mg of N-[(3R,6S)-6-(2,3-difluorophenyl)-2-oxo-1-(2,2,2-trifluoroethyl)azepan-3-yl]-4-(2-oxo-2,3-dihydro-1H-imidazo[4,5-b]pyridin-1-yl)piperidine-1-carboxamide, based on the equivalent weight. Suitable specific formulations comprise about 140, about 150, about 280, or about 300 mg of N-[(3R,6S)-6-(2,3-difluorophenyl)-2-oxo-1-(2,2,2-trifluoroethyl)azepan-3-yl]-4-(2-oxo-2,3-dihydro-1H-imidazo[4,5-b]pyridin-1-yl)piperidine-1-carboxamide.

The formulation may comprise about 25 to about 75% by weight of N-[(3R,6S)-6-(2,3-difluorophenyl)-2-oxo-1-(2,2,2-trifluoroethyl)azepan-3-yl]-4-(2-oxo-2,3-dihydro-1H-imidazo[4,5-b]pyridin-1-yl)piperidine-1-carboxamide as the active ingredient, for example about 35 to about 55% by weight. Typically, the composition may comprise about 50% by weight. Weight percent is determined from an equivalent weight measurement of the N-[(3R,6S)-6-(2,3-difluorophenyl)-2-oxo-1-(2,2,2-trifluoroethyl)azepan-3-yl]-4-(2-oxo-2,3-dihydro-1H-imidazo[4,5-b]pyridin-1-yl)piperidine-1-carboxamide potassium, as the hydrate, ethanolate (Form I or Form II, or mixtures thereof), or amorphous form.

It has been discovered that telcagepant does not effectively release from standard pharmaceutical formulations in vivo in the stomach of the patient, or in simulated gastric fluid. It is believed that the surface of standard formulations gel, thereby preventing water from penetrating into the formulation and inhibiting the release of the telcagepant active ingredient. In standard formulations the potassium salt converts to the neutral form, creating a relatively insoluble shell around the tablet. The shell effectively prevents dissolution of the drug.

As explained above, the invention is directed to solid dosage formulations of telcagepant comprising arginine, which have comparable bioavailability to liquid formulations of telcagepant. It is believed that arginine acts in the solid dosage formulation as a pharmaceutically acceptable basifying/dissolution enhancing agent. The basifying/dissolution enhancing agent enhances release of the active ingredient without significantly impacting other favorable properties of the formulation. The presence of the basifying/dissolution enhancing agent facilitates drug release from the formulation during tablet erosion and dissolution in the stomach, under acidic conditions.

Hence, the formulations of the invention include a “basifying/dissolution enhancer,” i.e., the monoaminodicarboxylic acid arginine ((NH₂CH—COOH(CH₂)₃—NH—CNH(NH₂)). The basifying/dissolution enhancing properties of arginine are believed to be due to its relatively high solubility, in combination with its high pKa and isolectric point. Arginine has a pKa of 2.03, 9.00 and 12. 1, and a pI (isoelectric point) of 10.76.

The amino acid basifying/dissolution enhancer acts to prevent or inhibit insoluble shell formation (neutral form) on the surface of the tablet during dissolution in the stomach or in simulated gastric fluid. Suitable formulations of the invention may comprise a basifying/dissolution enhancing amount of a basifying/dissolution enhancing agent (i.e. arginine). Suitable amounts are at least 5.0% basifying/dissolution enhancer agent, or at least 10.0% basifying/dissolution enhancer agent. Suitable amounts of the basifying/dissolution enhancer may be up to 90.0% basifying/dissolution enhancer agent (arginine). In other embodiments, a suitable amount is up to 50.0%, or up to 35.0%, or up to 30.0%. Suitable pharmaceutical formulations may comprise about 40.0% basifying/dissolution enhancer agent, about 30.0% basifying/dissolution enhancer agent, about 25.0% basifying/dissolution enhancer agent, about 20.0% basifying/dissolution enhancer agent, about 15.0% basifying/dissolution enhancer agent or about 10.0% basifying/dissolution enhancer agent.

In one embodiment of the invention, the solid dosage formulations are tablets.

The formulations of the invention may also comprise a pharmaceutically acceptable surfactant. As used herein, the term “pharmaceutically acceptable surfactant” or “surfactant” are used interchangeably, and refer to agents which reduce the surface tension of water by adsorbing at the liquid-gas interface. Surfactants are usually organic compounds that are amphiphilic, i. e., molecules comprising both hydrophobic groups and hydrophilic groups. Surfactants may generally be present in the amount of up to about 1 to 50% by weight of the formulation.

Surfactants suitable for use in the present invention may be classified as pharmaceutically acceptable anionic surfactants, cationic surfactants, amphoteric (amphipathic/amphophilic) surfactants, and non-ionic surfactants.

Preferred surfactants are non-ionic surfactants. The term “nonionic surfactant” is understood by one skilled in the art of pharmaceutical formulation to mean a class of surfactants which do not dissociate into ions in water. A preferred nonionic surfactant for the formulations of the invention is a polyoxypropylene block copolymer, also known as a “poloxamer,” comprising a central hydrophobic chain of polyoxypropylene and two hydrophilic chains of polyoxyethylene. Suitable poloxamers include Poloxamer 407.

The formulation may comprise up to 50% poloxamer, in some embodiments up to 10%, in other embodiments up to 7.5%. Suitable pharmaceutical formulations may comprise about 10.0% poloxamer, about 7.5% poloxamer, about 5.0%, or about 2.0% poloxamer.

Suitable pharmaceutically acceptable anionic surfactants include, for example, monovalent alkyl carboxylates, acyl lactylates, alkyl ether carboxylates, N-acyl sarcosinates, polyvalent alkyl carbonates, N-acyl glutamates, fatty acid-polypeptide condensates, sulfuric acid esters, alkyl sulfates (including sodium lauryl sulfate (SLS)), ethoxylated alkyl sulfates, ester linked sulfonates (including docusate sodium or dioctyl sodium succinate (DSS)), alpha olefin sulfonates, and phosphated ethoxylated alcohols.

Suitable pharmaceutically acceptable cationic surfactants include, for example, monoalkyl quaternary ammonium salts, dialkyl quaternary ammonium compounds, amidoamines, and aminimides.

Suitable pharmaceutically acceptable amphoteric (amphipathic/amphophilic) surfactants, include, for example, N-substituted alkyl amides, N-alkyl betaines, sulfobetaines, and N-alkyl β-aminoproprionates.

Other suitable surfactants for use in conjunction with the present invention include polyethyleneglycols as esters or ethers. Examples include polyethoxylated castor oil, polyethoxylated hydrogenated castor oil, or polyethoxylated fatty acid from castor oil or polyethoxylated fatty acid from hydrogenated castor oil. Commercially available surfactants that can be used are known under trade names Cremophor, Myrj, Polyoxyl 40 stearate, Emerest 2675, Lipal 395 and PEG 3350.

In still other embodiments, the formulation comprises a pharmaceutically acceptable disintegrant. Disintegrants are substances added to pharmaceutical tablets that facilitate the breakup or disintegration of the tablet after administration. Suitable disintegrants are starches (including corn starch and potato starch), clays, celluloses, aligns, gums and cross-linked polymers. Suitable disintegrants include the class of disintegrants known as “super disintegrants,” which may typically be used in lower amounts than other disintegrants. Exemplary classes of super disintegrants include croscarmellose, cross-linked polyvinyl pyrrolidine (also known as crospovidone) and sodium starch glycosate.

The disintegrant (including super disintegrants) may be present in the amount of up to about 20% by weight of the formulation.

In still other embodiments, the formulation comprises additional pharmaceutically acceptable excipients, including, for example, fillers, glidants, lubricants, coloring agents, coating agents and waxes.

Fillers are added to provide bulk to formulations, in order to ease handling and processing. Suitable pharmaceutically acceptable fillers for use in the invention include mannitol, AVICEL, non-lactose fillers, and other fillers that do not interact with amine groups. Glidants improve the flow characteristics of the powder. Suitable glidants for use in the invention include colloidal silicon dioxide and talc. Glidants are typically present in the formulation in the amount of up to about 1% by weight. In some embodiments of the invention, the lubricant is present in the amount of up to 0.5% by weight.

Lubricants also reduce interparticle friction, and facilitate the ejection of tablets from the die. Exemplary lubricants for use in the invention include talc, magnesium stearate (intragranular and/or extragranular), calcium stearate, stearic acid, glyceryl behanate, hydrogenated vegetable oil and polyethylene glycol. Lubricants are typically present in the formulation in the amount of up to 2% by weight. In some embodiments of the invention, the lubricant is present in the amount of up to 1% by weight, or up to 0.5% by weight. Coloring agents improve the aesthetics of the drug formulations, and help to distinguish and identify formulations during manufacturing. Coloring agents useful in the invention include any of the colorants approved by the Food and Drug Administration for use in pharmaceutical formulations.

Film coating agents may also be used to coat the formulation. Suitable film coating agents include OPADRY and OPADRY II (with a mixture of various coloring agents), which are manufactured by Colorcon, Inc. These are hydroxypropyl cellulose, HPMC 2910/hypromellose 6 cp base and polyvinyl alcohol base coating formulations.

The invention is also directed to a method of treating headaches, comprising administering to a patient the solid dosage formulation of the invention.

Another embodiment of the present invention is directed to a method for the treatment, control, amelioration, or reduction of risk of a disease or disorder in which the CGRP receptor is involved (such as headaches) in a patient, comprising administering to the patient a formulation of the invention.

In one embodiment, the solid dosage formulations of the invention provide Cmax in the blood of at least 2.75 μM. In other embodiments, the solid dosage formulations of the invention provide Cmax in the blood of at least at least 3.0 μM. In particular embodiments, the desirable Cmax values listed above are achieved for formulations comprising about 280 mg of the telcagepant active ingredient, and for formulations comprising about 300 mg of the telcagepant active ingredient.

In one embodiment, the solid dosage formulations of the invention achieve a Tmax at a time point of no more than 1.0 hour after administration. In another embodiment, the solid dosage formulations of the invention achieve a Tmax at a time point of no more than 1.25 hour after administration. In still another embodiment, the solid dosage formulations of the invention achieve a Tmax at a time point of no more than about 1.5 hours after administration.

In one embodiment, the solid dosage formulations of the invention demonstrate an AUC_{0-Tmax} in the blood of no more than 2.5 μM hr. In other embodiments, the solid dosage formulations of the invention demonstrate an AUC_{0-Tmax} in the blood of no more than 2.0 μM hr.

In one embodiment, the solid dosage formulations of the invention demonstrate an AUC₀₋₂ hr in the blood of no more than 5.5 μM hr. In other embodiments, the solid dosage formulations of the invention demonstrate an AUC₀₋₂ hr in the blood of no more than 4.5 μM hr. In one embodiment, the solid dosage formulations of the invention demonstrate an AUC₀₋₄ hr in the blood of no more than 10.0 ∥M hr. In other embodiments, the solid dosage formulations of the invention demonstrate an AUC₀₋₄ hr in the blood of no more than 9.0 μM hr.

In one embodiment, the solid dosage formulations of the invention demonstrate an AUC_{0-∞} in the blood of no more than 15.5 μM hr. In other embodiments, the solid dosage formulations of the invention demonstrate an AUC_{0-∞} in the blood of no more than 15.0 μM hr.

Exemplary formulations are shown in Table 1 below:

TABLE 1

| | Tablet 1 - 280 mg | | Tablet 2 - 140 mg | | Tablet 3 - 50 mg | |
|---|---|---|---|---|---|---|
| | telcagepant | | telcagepant | | telcagepant | |
| Ingredients | (weight percent) | | (weight percent) | | (weight percent) | |
| Telcagepant | 50 | | 50 | | 50 | |
| Potassium | | | | | | |
| Poloxamer 407 | 5 | | 5 | | 5 | |
| Arginine | 25 | | 25 | | 25 | |
| Mannitol | 14 | | 14 | | 14 | |
| Crospovidone | 3.5 | | 3.5 | | 3.5 | |
| Silicone | 0.5 | | 0.5 | | 0.5 | |
| dioxide | | | | | | |
| Magnesium | 2 | | 2 | | 2 | |
| stearate | | | | | | |
| Film coat | 3.0 | | 3.7 | | 4.58 | |
| Wax | 0.01 | | 0.01 | | 0.01 | |
| CORE | 664 | mg | 332 | mg | 115 | mg |
| TABLET | | | | | | |
| WEIGHT | | | | | | |

The core tablet weight is calculated according to a salt converstion factor, as known to one skilled in the art (e.g., 1.1494 g of telcagepant potassium salt equals 1 g of neutral telcagepant).

### Definitions

As used herein, the terms “telcagepant” and “compound 1” are used interchangeably, and mean the compound N-[(3R,6S)-6-(2,3-Difluorophenyl)-2-oxo-1-(2,2,2-trifluoroethyl)azepan-3-yl]-4-(2-oxo-2,3-dihydro-1H-imidazo[4,5-b]pyridin-1-yl)piperidine-1-carboxamide:

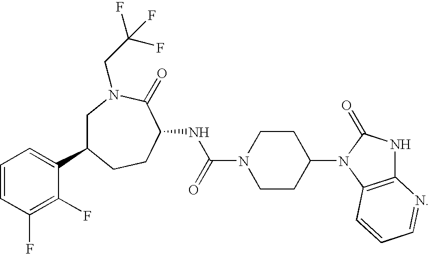

The USAN council has adopted the term “telcagepant potassium” to refer to the potassium salt of N-[(3R,6S)-6-(2,3-Difluorophenyl)-2-oxo-1-(2,2,2-trifluoroethyl)azepan-3-yl]-4-(2-oxo-2,3-dihydro-1H-imidazo[4,5-b]pyridin-1-yl)piperidine-1-carboxamide with ethanol. However, as used herein, the term “telcagepant potassium” refers to all forms or solvates of the potassium salt of N-[(3R,6S)-6-(2,3-Difluorophenyl)-2-oxo-1-(2,2,2-trifluoroethyl)azepan-3-yl]-4-(2-oxo-2,3-dihydro-1H-imidazo[4,5-b]pyridin-1-yl)piperidine-1-carboxamide (compound 1A):

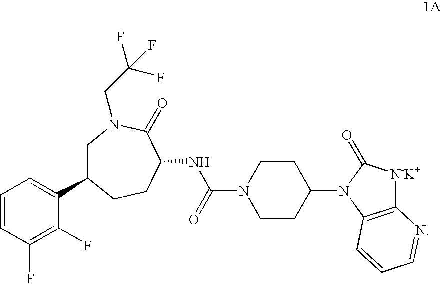

As used herein, the term “telcagepant potassium ethanolate” refers to the ethanolate of N-[(3R,6S)-6-(2,3-Difluorophenyl)-2-oxo-1-(2,2,2-trifluoroethyl)azepan-3-yl]-4-(2-oxo-2,3-dihydro-1H-imidazo[4,5-b]pyridin-1-yl)piperidine-1-carboxamide potassium (compound 1C):

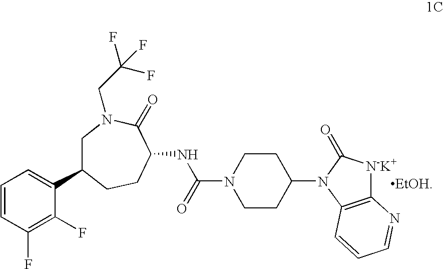

As used herein, the term “telcagepant potassium hydrate” refers to the hydrate of N-[(3R,6S)-6-(2,3-Difluorophenyl)-2-oxo-1-(2,2,2-trifluoroethyl)azepan-3-yl]-4-(2-oxo-2,3-dihydro-1H-imidazo[4,5-b]pyridin-1-yl)piperidine-1-carboxamide potassium (compound 1B):

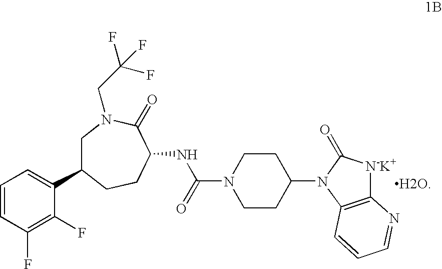

As used herein, the terms “treatment,” “treating,” and the like, refer to obtaining a desired pharmacologic and/or physiologic effect. The effect may be prophylactic in terms of completely or partially preventing a disease, disorder or condition, or symptom thereof and/or may be therapeutic in terms of a partial or complete cure for a disease, disorder or condition, and/or adverse affect attributable thereto. “Treatment”, as used herein, covers any treatment of a disease, disorder or condition, in a mammal, particularly in a human, and includes: (a) preventing the disease, disorder or condition, from occurring in a subject which may be predisposed to the disease, disorder or condition, but has not yet been diagnosed as having it; (b) inhibiting the disease, disorder or condition, i.e. arresting its development; and (c) relieving the disease, disorder or condition, i.e., causing regression.

The terms “individual,” “subject,” and “patient,” used interchangeably herein, refer to a mammal, including, but not limited to, murines, simians, humans, mammalian farm animals, mammalian sport animals, and mammalian pets. Preferably, the patient is a human (male or female).

A “therapeutically effective amount” or “effective amount” means the amount of a telcagepant, or salt or solvate thereof (e.g., the amount of telcagepant, or a salt or solvate thereof) that, when administered to a mammal or other subject for treating a disease, is sufficient to effect such treatment for the disease. The “therapeutically effective amount” will vary depending on the compound, the disease and its severity and the age, weight, etc., of the subject to be treated.

The term “pharmaceutically acceptable,” when used alone in such phrases as “pharmaceutically acceptable excipient,” “pharmaceutically acceptable diluent,” “pharmaceutically acceptable carrier,” and “pharmaceutically acceptable adjuvant”, mean an excipient, diluent, carrier, adjuvant or similar materials that are useful in preparing a pharmaceutical formulations that are generally safe, non-toxic and neither biologically nor otherwise-undesirable, and include an excipient, diluent, carrier, and adjuvant that is acceptable for veterinary use as well as human pharmaceutical use. “Pharmaceutically acceptable” materials are, within the scope of sound medical judgment, suitable for contact with the tissues of human beings and animals without excessive toxicity, irritation, allergic response, or other problem complications commensurate with a reasonable benefit/risk ratio. In some embodiments, the term “pharmaceutically acceptable” means approved by a regulatory agency of the Federal or a state government or listed in the U.S. Pharmacopoeia or other generally recognized international pharmacopoeia for use in animals, and more particularly in humans.

“A pharmaceutically acceptable excipient,” or pharmaceutically acceptable “diluent,” “carrier” or “adjuvant,” as used in the specification and claims, includes both one and more than one such excipient, diluent, carrier, or adjuvant. An “excipients,” “diluent,” “carrier” or “adjuvant” refers to a substance that is used in the formulation of solid dosage pharmaceutical formulations, and, by itself, generally has little or no therapeutic value. Various excipients, diluents, carrier or adjuvants can be used in the invention, including those described in Remington: The Science and Practice of Pharmacy, 21^{st} Ed., pp. 317-318 (2006). These include, but are not limited to, surfactants, disintegrants, fillers, antioxidants, anti-bacterial agents that prevent the decay of the formulation itself as opposed to those exhibiting a therapeutic effect, preservatives, chelating agents, buffering agents, glidants, lubricants, agents for adjusting toxicity, colorings, flavorings and diluting agents, emulsifying and suspending agents, and other substances with pharmaceutical applications.

The term “solid unit dosage form,” as used herein, refers to physically discrete, solid units suitable as unitary dosages for human and animal subjects, each unit containing a predetermined quantity of compounds of the present invention calculated in an amount sufficient to produce the desired effect in association with a pharmaceutically acceptable diluent, carrier or vehicle. Exemplary “solid unit dosage forms” are tablets, capsules, pills, troches, cachets and pellets. The solid dosage formulations of the invention are designed for use by an oral route of administration.

As used herein, a “pharmaceutical formulation” is meant to encompass a composition suitable for oral administration to a subject, such as a mammal, especially a human. In general a “pharmaceutical formulation” is sterile, and generally free of contaminants that are capable of eliciting an undesirable response within the subject (e.g., the compound(s) in the pharmaceutical composition is pharmaceutical grade).

### Forms of Telcagepant

Ethanolate

As noted above, the telcagepant potassium ethanolate, and methods of synthesis, are disclosed in International Application WO 2007/120592. Methods of manufacturing Form I of the telcagepant potassium ethanolate is disclosed in International Application WO 2007/120592, Examples 3-6. Form II of the ethanolate has been observed to form during manufacturing when no seeds (crystals) of Form I were added to the solution of telcagepant potassium.

The potassium salt ethanolate Form I exhibits diffraction peaks corresponding to d-spacings of 8.27, 4.01, and 3.32 angstroms. The potassium salt ethanolate Form I is further characterized by the d-spacings of 16.52, 7.55, and 7.02 angstroms. The potassium salt ethanolate Form I is even further characterized by the d-spacings of 5.52, 5.08, and 4.63 angstroms.

The potassium salt ethanolate Form II exhibits characteristic diffraction peaks corresponding to d-spacings of 11.62, 7.80, and 4.92 angstroms. The potassium salt ethanolate Form II is further characterized by the d-spacings of 4.55, 4.31, and 4.1 1 angstroms. The potassium salt ethanolate Form II is even further characterized by the d-spacings of 3.85, 3.55 and 2.88 angstroms.

Form I is characterized by solid-state carbon-13 NMR spectra peaks of 109.1 ppm, 55.8 ppm and 54.6 ppm.

The Raman spectra of the Form I telcagepant potassium salt ethanolate is characterized by peaks (cm⁻¹) of 646.3, 707.4, 761.5, 832.9, 1063.3, 1365.5, 1402.0, 1445.7, 1455.3

Hydrate

The telcagepant potassium hydrate, and methods of synthesis, are disclosed in International Application WO 2007/120592.

The potassium salt hydrate exhibits characteristic diffraction peaks corresponding to d-spacings of 16.96, 8.50, and 4.26 angstroms. The potassium salt hydrate is further characterized by the d-spacings of 7.41, 6.88, and 3.79 angstroms. The potassium salt hydrate is even further characterized by the d-spacings of 5.00, 3.41 and 3.06 angstroms.

The potassium salt hydrate is characterized by solid-state carbon-13 NMR spectra peaks of 126.1 ppm, 54.4 ppm and 36.6 ppm.

The Raman spectra of the potassium salt hydrate is characterized by peaks (cm⁻¹) of 646.8, 707.0, 753.7, 832.7, 1064.7, 1364.3, 1403.0, 1441.0

Amorphous Form

As used herein, the term “amorphous form” refers to a chemically and physically stable amorphous, non-crystalline form of telcagepant potassium. The amorphous form does not convert to crystalline form in storage, but is hygroscopic and absorbs water if not protected from humidity.

The amorphous form may be obtained by spray drying of the potassium salt of telcagepant in an organic solution without the addition of any polymers. During the spray drying process, the liquid feedstock is atomized into a spray of droplets of micron size and the evaporation of solvent occurs rapidly upon contacting the droplets with a hot processing gas in a drying chamber. The formation of dry particles proceeds under controlled temperature and gas flow conditions. This rapid evaporation of the organic solvent results in a formation of amorphous drug. Suitable organic solutions include methanol and acetone.

Alternatively, the amorphous form may be prepared by heating the telcagepant potassium salt ethanolate, and passing wet nitrogen gas over the ethanolate.

The amorphous form may be obtained by an impinging jet process, in which a concentrated solution of telcagepant in isopropyl acetate is mixed quickly with an anti-solvent (for example, heptane), thereby forming the amorphous form as a precipitate. The addition of a small amount of water to the feed stream improves the morphology of the particles of the amorphous form.

The morphology, particle size distribution and surface area differ according to how the amorphous form is made. The amorphous form made by spray drying is typically smaller, and is a relatively cohesive material. The spray-dried amorphous form is chemically stable at 40° C./75% relative humidity, for six weeks.

The amorphous form produced by spray drying has a mean particle size of less than 15 μm, often less than 10 μm; a density (g/cm³) of 0.20 or less, often 0.15 or less; a Carr's Index (percentability compression) of 35-45%; a Hausner ratio of about 1.64; and a surface area (m²/g) of 3.0 or less, often 2.5 or less, often 2.0 or less.

Carr's index is frequently used in pharmaceutical technology as an indication of the flowability of a powder. See Mark Gibson, “Pharmaceutical Preformulation and Formulation: A Practical Guide from Candidate Drug Selection to Commercial Dosage Form,” Boca Raton: CRC Press. (2001).

The Hausner ratio is a measure of the flowability of a powder.

The amorphous form made by solution precipitation has a broader particle size distribution, high surface area. It is expected that the amorphous form made by solution precipitation will be a porous material.

The amorphous form produced by precipitation has a mean particle size of less than 150 μm, often less than 125 μm, often less than 110 μm; a Carr's Index (percentability compression) of 25-30%; a Hausner ratio of about 1.38 or more; and a surface area (m²/g) of 50-100 m²/g, often 70-90 m²/g.

The amorphous potassium salt demonstrates a heat capacity change in the reversing heat flow curve with a midpoint temperature of 189.00° C., which corresponds to the glass transition of amorphous potassium salt.

The amorphous form of the potassium salt is characterized by solid-state carbon-13 NMR spectra peaks of 126.0 ppm, 53.7 ppm and 29.1 ppm.

The Raman spectra of the amorphous potassium salt is characterized by peaks (cm⁻¹) of 646.8, 706.8, 752.3, 832.4, 1063.6, 1365.2, 1437.6.

Manufacture of Formulations

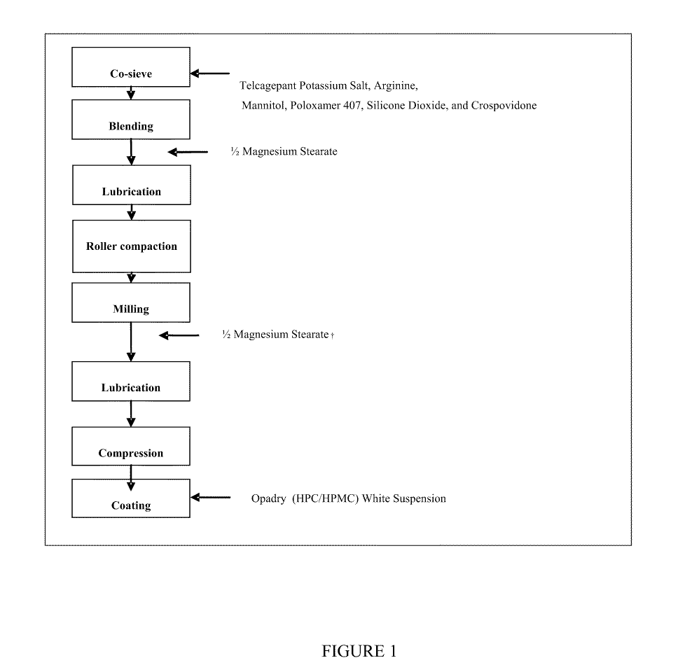

The formulations of the invention may be prepared by a dry granulation method. The tablet manufacturing process is essentially the same, for all drug substance forms (potassium salt hydrate, potassium salt ethanolate (Form I or Form II, or mixtures thereof), potassium salt amorphous) of N-[(3R,6S)-6-(2,3-difluorophenyl)-2-oxo-1-(2,2,2-trifluoroethyl)azepan-3-yl]-4-(2-oxo-2,3-dihydro-1H-imidazo[4,5-b]pyridin-1-yl)piperidine-1-carboxamide. The manufacturing process flow diagram of FIG. 1 describes a suitable process for manufacturing a solid dosage formulation of the invention.

Alternatively, a wet granulation process may be used. Wet granulation methods for producing pharmaceutical tablets are well known to those skilled in the art. Typically, wet grantulation processes involve the steps of weighing, mixing, granulating, screening the damp mass, drying, dry screening, lubricating and compressing the mass into a tablet. The mixing steps occur in a blender, such as a twin shell blender, double cone blender or ribbon blender, or in a planetary mixer or a high speed/high shear mixer.

The formulations may also be prepared by a fluid bed granulation process.

Dry granulation, wet granulation and fluid bed granulation processes are described in Remington's “The Science and Practice of Pharmacy,” ₂₁st ed. (2006), pp. 896-901.

### Dosages and Uses of the Formulation of the Invention

The ability of the formulations of the invention to act as CGRP antagonists makes them useful pharmacological agents for disorders that involve CGRP in humans and animals, but particularly in humans.

The formulations of the present invention have utility in treating, preventing, ameliorating, controlling or reducing the risk of one or more of the following conditions or diseases: headache; migraine; cluster headache; chronic tension type headache; pain; chronic pain; neurogenic inflammation and inflammatory pain; neuropathic pain; eye pain; tooth pain; diabetes; non-insulin dependent diabetes mellitus; vascular disorders; inflammation; arthritis; bronchial hyperreactivity, asthma; shock; sepsis; opiate withdrawal syndrome; morphine tolerance; hot flashes in men and women; allergic dermatitis; encephalitis; brain trauma; epilepsy; neurodegenerative diseases; skin diseases; neurogenic cutaneous redness, skin rosaceousness and erythema; tinnitus; inflammatory bowel disease, irritable bowel syndrome, cystitis; and other conditions that may be treated or prevented by antagonism of CGRP receptors. Of particular importance is the acute or prophylactic treatment of headache, including migraine and cluster headache.

The dosage of the potassium salt of telcagepant (or the hydrate or ethanolate or amorphous form thereof), administered will be dependent upon the age, health, and weight of the recipient, kind of concurrent treatment, if any, the frequency of treatment, and the nature of the effect desired. The formulations of the present invention can contain a quantity of the potassium salt of telcagepant (or the hydrate or ethanolate thereof, or an amorphous form thereof), according to this invention in an amount effective to treat the condition, disorder or disease of the subject being treated. One of ordinary skill in the art will appreciate that a method of administering pharmaceutically effective amounts of the potassium salt of telcagepant (or the hydrate or ethanolate thereof (Form I or Form II, or mixtures thereof), or an amorphous form thereof), to a patient in need thereof can be determined empirically, or by standards currently recognized in the medical arts. It will be understood that, when administered to, for example, a human patient, the total daily dosage of the agents of the formulations of the present invention will be decided within the scope of sound medical judgment by the attending physician.

The dosages of the invention are described according to the amount of available telcagepant as the active ingredient, in its neutral form as N-[(3R,6S)-6-(2,3-difluorophenyl)-2-oxo-1-(2,2,2-trifluoroethyl)azepan-3-yl]-4-(2-oxo-2,3-dihydro-1H-imidazo[4,5-b]pyridin-1-yl)piperidine-1-carboxamide. As is understood by the skilled artisan, the amount of active ingredient is calculated according to a conversion factor, calculated based on the form of telcagepant used in the formulation the potassium salt ethanolate, the potassium salt hydrate, the potassium salt amorphous), and other elements such as the assay and purity ( amounts of water, ethanol, solvents or other impurities) of the manufactured lot. An exemplary conversion factor for the ethanolate is 1. 1494 g ethanolate is equal to 1.0 g active ingredient (or the neutral form). An exemplary conversion factor for the hydrate is 1. 157 g hydrate is equal to 1. 0 g active ingredient (or the neutral form). An exemplary conversion factor for the amorphous form is 1.067 g amorphous form is equal to 1.0 g active ingredient (or the neutral form).

Thus, a 100 mg unit dose formulation will include 115.2 mg ethanolate (if telcagepant is in the form of the ethanolate of the potassium salt), 115.7 mg hydrate (if telcagepant is in the form of the hydrate of the potassium salt), or 106.7 mg amorphous (if telcagepant is in the amorphous form of the potassium salt).

In the treatment, prevention, control, amelioration, or reduction of risk of conditions which require antagonism of CGRP receptor activity an appropriate dosage level will generally be about 0.01 to 500 mg of the telcagepant active ingredient, per kg patient body weight per day which can be administered in single or multiple doses. A suitable dosage level may be about 0.01 to 250 mg/kg per day, about 0.05 to 100 mg/kg per day, or about 0.1 to 50 mg/kg per day. The telcagepant active ingredient (in the form of the potassium salt hydrate, ethanolate or amorphous form) may be administered on a regimen of 1 to 4 times per day, or may be administered once or twice per day. This dosage regimen may be adjusted to provide the optimal therapeutic response.

The amount of telcagepant active ingredient may be combined with the carrier materials to produce a single dosage form will vary depending upon the host treated and the particular mode of administration. For example, a formulation intended for the oral administration to humans may conveniently contain from about 0.005 mg to about 2.5 g of telcagepant, compounded with an appropriate and convenient amount of carrier material. Unit dosage forms will generally contain between from about 0.005 mg to about 1000 mg of telcagepant, typically 0.005 mg, 0.01 mg, 0.05 mg, 0.25 mg, 1 mg, 5 mg, 25 mg, 50 mg, 100 mg, 150 mg, 200 mg, 250 mg, 300 mg, 350 mg, 400 mg, 500 mg, 600 mg, 800 mg or 1000 mg, administered once, twice or three times a day. Preferred unit dosage forms are from 100 to 200 mg, or from 250 mg to 350 mg.

The specific therapeutically effective dose level of the telcagepant active ingredient for any particular patient will depend upon a variety of factors: the type and degree of the cellular response to be achieved; activity of the specific agent used; the specific agents used; the age, body weight, general health, gender and diet of the patient; the time of administration, route of administration, and rate of excretion of the agent; the duration of the treatment; drugs used in combination or coincidental with the specific agent; and like factors well known in the medical arts. For example, it is well within the skill of the art to start doses of telcagepant at levels lower than those required to achieve the desired therapeutic effect and to gradually increase the dosages until the desired effect is achieved.

Combination Therapies with the Formulation of the Invention

The formulations of the invention may be used in conjunction with an anti-inflammatory or analgesic agent or an anti-migraine agent, such as an ergotamine or 5-HT₁ agonists, especially a 5-HT_{1B/1D} agonist, for example sumatriptan, naratriptan, zolmitriptan, eletriptan, almotriptan, frovatriptan, donitriptan, and rizatriptan; a cyclooxygenase inhibitor, such as a selective cyclooxygenase-2 inhibitor, for example rofecoxib, etoricoxib, celecoxib, valdecoxib or paracoxib; a non-steroidal anti-inflammatory agent or a cytokine-suppressing anti-inflammatory agent, for example with a compound such as aspirin, ibuprofen, ketoprofen, fenoprofen, naproxen, indomethacin, sulindac, meloxicam, piroxicam, tenoxicam, lomoxicam, ketorolac, etodolac, mefenamic acid, meclofenamic acid, flufenamic acid, tolfenamic acid, diclofenac, oxaprozin, apazone, nimesulide, nabumetone, tenidap, etanercept, tolmetin, phenylbutazone, oxyphenbutazone, diflunisal, salsalate, olsalazine or sulfasalazine and the like; or a steroidal analgesic. Similarly, the instant compounds may be administered with a pain reliever such as acetaminophen, phenacetin, codeine, fentanyl, sufentanil, methadone, acetyl methadol, buprenorphine or morphine.

Additionally, the formulations of the invention may be used in conjunction with an interleukin inhibitor, such as an interleukin-1 inhibitor; an NK-1 receptor antagonist, for example aprepitant; an NMDA antagonist; an NR2B antagonist; a bradykinin-1 receptor antagonist; an adenosine A1 receptor agonist; a sodium channel blocker, for example lamotrigine; an opiate agonist such as levomethadyl acetate or methadyl acetate; a lipoxygenase inhibitor, such as an inhibitor of 5-lipoxygenase; an alpha receptor antagonist, for example indoramin; an alpha receptor agonist; a vanilloid receptor antagonist; an mGluR5 agonist, antagonist or potentiator; a GABA A receptor modulator, for example acamprosate calcium; nicotinic antagonists or agonists including nicotine; muscarinic agonists or antagonists; a selective serotonin reuptake inhibitor, for example fluoxetine, paroxetine, sertraline, duloxetine, escitalopram, or citalopram; a tricyclic antidepressant, for example amitriptyline, doxepin, protriptyline, desipramine, trimipramine, or imipramine; a leukotriene antagonist, for example montelukast or zafirlukast; an inhibitor of nitric oxide or an inhibitor of the synthesis of nitric oxide.

Also, the formulations of the invention may be used in conjunction with ergot alkaloids, for example ergotamine, ergonovine, ergonovine, methylergonovine, metergoline, ergoloid mesylates, dihydroergotamine, dihydroergocornine, dihydroergocristine, dihydroergocryptine, dihydro-I-ergocryptine, dihydro-θ-ergocryptine, ergotoxine, ergocornine, ergocristine, ergocryptine, I-ergocryptine, θ-ergocryptine, ergosine, ergostane, bromocriptine, or methysergide.

Additionally, the formulations of the invention may be used in conjunction with a beta-adrenergic antagonist such as timolol, propanolol, atenolol, or nadolol, and the like; a MAO inhibitor, for example phenelzine; a calcium channel blocker, for example flunarizine, nimodipine, lomerizine, verapamil, nifedipine, prochlorperazine or gabapentin; neuroleptics such as olanzapine and quetiapine; an anticonvulsant such as topiramate, zonisamide, tonabersat, carabersat or divalproex sodium; an angiotensin II antagonist, for example losartan and candesartan cilexetil; an angiotensin converting enzyme inhibitor such as lisinopril; or botulinum toxin type A.

The formulations of the invention may be used in conjunction with a potentiator such as caffeine, an H2-antagonist, simethicone, aluminum or magnesium hydroxide; a decongestant such as phenylephrine, phenylpropanolamine, pseudoephedrine, oxymetazoline, epinephrine, naphazoline, xylometazoline, propylhexedrine, or levo-desoxy-ephedrine; an antitussive such as codeine, hydrocodone, caramiphen, carbetapentane, or dextromethorphan; a diuretic; a prokinetic agent such as metoclopramide or domperidone, and a sedating or non-sedating antihistamine.

In a particularly preferred embodiment, the formulations of the invention are used in conjunction with an anti-migraine agent, such as: an ergotamine; a 5-HT₁ agonist, especially a 5-HT_{1B/1D} agonist, in particular, sumatriptan, naratriptan, zolmitriptan, eletriptan, almotriptan, frovatriptan, donitriptan and rizatriptan; and a cyclooxygenase inhibitor, such as a selective cyclooxygenase-2 inhibitor, in particular, rofecoxib, etoricoxib, celecoxib, meloxicam, valdecoxib or paracoxib.

The above combinations include formulations of the invention not only with one other active compound, but also with two or more other active compounds. Likewise, formulations of the invention may be used in combination with other drugs that are used in the prevention, treatment, control, amelioration, or reduction of risk of the diseases or conditions for which compounds of the present invention are useful. Such other drugs may be administered, by a route and in an amount commonly used therefor, contemporaneously or sequentially with a compound of the present invention.

In such combinations the formulation of the present invention and other active agents may be administered separately or in conjunction. In addition, the administration of one element may be prior to, concurrent to, or subsequent to the administration of other agent(s), and via the same or different routes of administration.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

“Optional” or “optionally” means that the subsequently described event, circumstance, feature, or element may, but need not, occur, and that the description includes instances where the event or circumstance occurs and instances in which it does not.

It must be noted that as used herein and in the appended claims, the singular forms “a,” “and,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

### Examples

### Example 1

### Amorphous Form of Telcagepant Potassium

A sample of the potassium salt ethanolate of telcagepant was dissolved in methanol at 12 weight %. The solution was spray dried in SD-Micro, manufactured by Niro A/S, of Denmark, at the following conditions:

Processing gas rate at 30 kg/hr

Atomization rate at 2 kg/hr

Feed rate at 15 mL/min

Inlet temperature: 136° C.

Outlet temperature: 65° C.

The resulting powder was measured by x-ray powder diffraction spectra, using X'pert X-ray diffractometer, manufactured by Philips, Inc. The diffraction angle was run from 4 to 40°. A single amorphous formation was indicated by the profile of a broad halo.

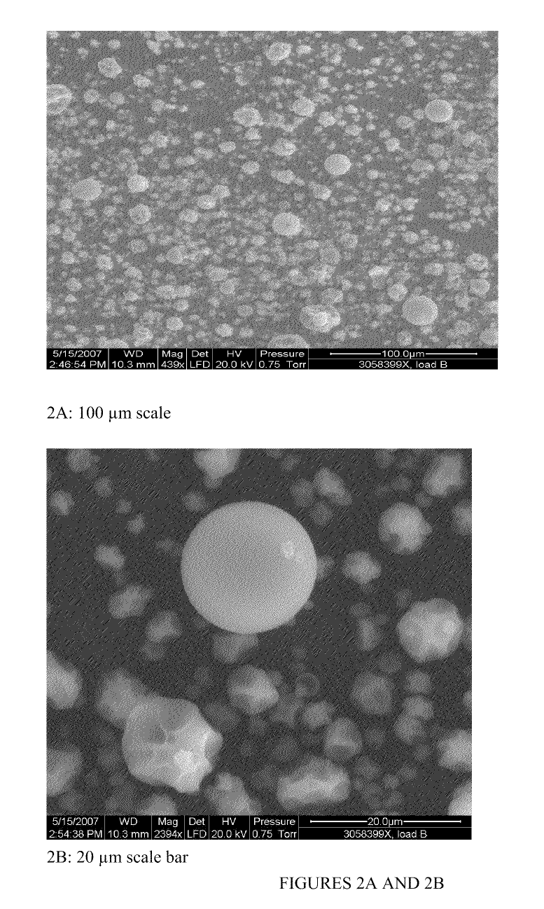

Photographs of the resulting powder are shown in FIGS. 2A (100 μm scale bar) and 2B (20 μm scale bar).

The resulting powder was characterized as having a mean particle size of 7 μm. 95% of the powder had a particle size of less than 18 μm, and 10% had a particle size of less than 2 μm. The density of the powder was measured “loose” at 0.11 g/cm³, and “tapped” at 0.18 g/cm³.

Carr's density was measured at 39%, and the Hausner ratio was 1.64. The surface area was 1.5 m²/g.

Water content at 25° C./75% relative humidity was determined to be 18%.

### Example 2

### Precipitation Method of the Telcagepant Potassium Amorphous Form

A concentrated stream of the potassium salt of telcagepant is prepared in ethyl acetate or other good solvent (e.g, THF), in the range: 40-300 mg/ml. Water may be added to the concentrated M solution such that the water content is between 0-2 wt %. The water aids the formation of three dimensional particles that are easily filtered. Amorphous potassium salt of telcagepant is then precipitated with an “impinging jet” technique by contacting the concentrated stream with heptane or other anti-solvent (e.g. cyclohexane) in a ratio of 1 volume of concentrated batch to 2 or more volumes of heptane using an impinging jet contacting apparatus. In this apparatus, the concentrated stream is continuously fed with a syringe pump into small volume, and at the same time the anti-solvent is added to this volume with a syringe pump. The product precipitates after the streams are contacted and the resulting product slurry is collected in a collection flask. In this way the apparatus appears as a “T” shape with inlets for the batch and heptane, and an outlet for the product slurry. The slurry is filtered and washed with heptane. The product is then dried in a vacuum oven at 40-50° C.

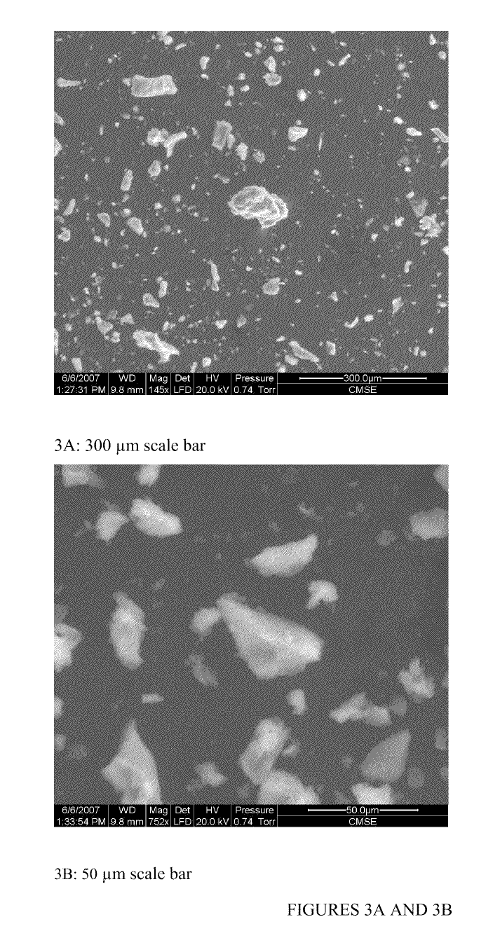

Photographs of a powder produced by the process of Example 3 is shown in FIGS. 3A (300 μm scale bar) and 3B (50 μm scale bar).

The resulting powder was characterized as having a mean particle size of 99 μm. 95% of the powder had a particle size of less 296 μm, and 10% had a particle size of less than 11 μm. The density of the powder was measured “loose” at 0.24 g/cm³, and “tapped” at 0.33 g/cm³.

Carr's density was measured at 27%, and the Hausner ratio was 1.38. The surface area was 80.6 m²/g.

Water content at 25° C./75% relative humidity was determined to be about 18%.

### Example 3

### X-Ray Powder Diffraction Studies of Telcagepant Potassium Forms

X-ray powder diffraction studies are widely used to characterize molecular structures, crystallinity, and polymorphism. The X-ray powder diffraction patterns of the potassium salt ethanolate Form I and Form II, and potassium salt hydrate were generated on a Philips Analytical X'Pert PRO X-ray Diffraction System with PW3040/60 console. A PW3373/00 ceramic Cu LEF X-ray tube K-Alpha radiation was used as the source.

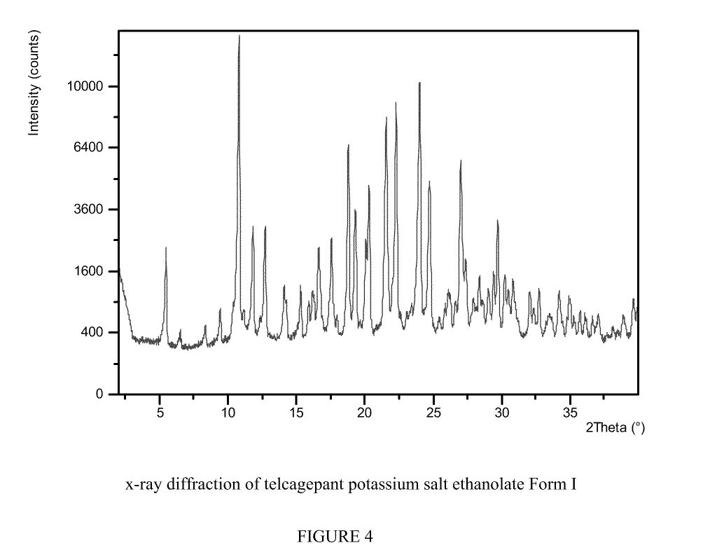

FIG. 4 shows the X-ray powder diffraction pattern of the potassium salt ethanolate Form I. The potassium salt ethanolate Form I exhibited characteristic diffraction peaks corresponding to d-spacings of 8.27, 4.01, and 3.32 angstroms. The potassium salt ethanolate Form I was further characterized by the d-spacings of 16.52, 7.55, and 7.02 angstroms. The potassium salt ethanolate Form I was even further characterized by the d-spacings of 5.52, 5.08, and 4.63 angstroms.

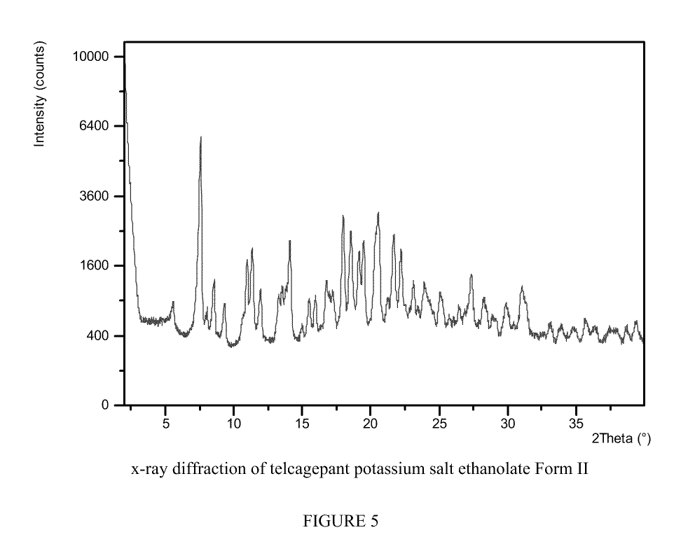

FIG. 5 shows the X-ray powder diffraction pattern of the potassium salt ethanolate Form II. The potassium salt ethanolate Form II exhibited characteristic diffraction peaks corresponding to d-spacings of 11.62, 7.80, and 4.92 angstroms. The potassium salt ethanolate Form II was further characterized by the d-spacings of 4.55, 4.31, and 4.11 angstroms. The potassium salt ethanolate Form II was even further characterized by the d-spacings of 3.85, 3.55 and 2.88 angstroms.

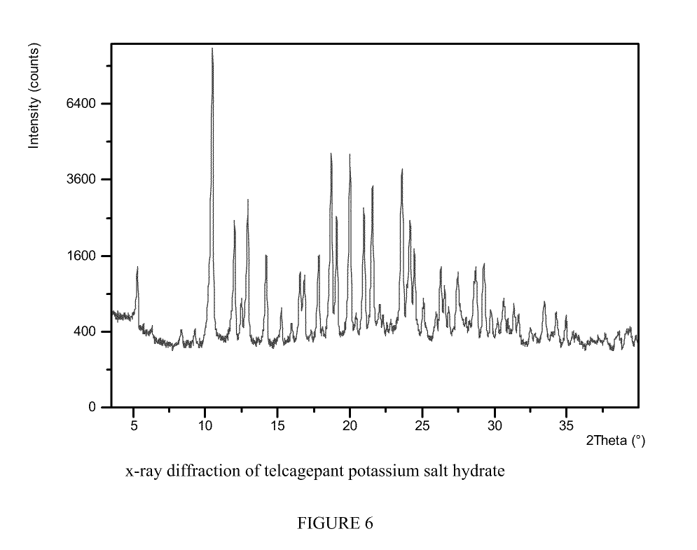

FIG. 6 shows the X-ray powder diffraction pattern of the potassium salt hydrate. The potassium salt hydrate exhibited characteristic diffraction peaks corresponding to d-spacings of 16.96, 8.50, and 4.26 angstroms. The potassium salt hydrate was further characterized by the d-spacings of 7.41, 6.88, and 3.79 angstroms. The potassium salt hydrate was even further characterized by the d-spacings of 5.00, 3.41 and 3.06 angstroms.

### Example 4

### Modulated DSC Studies of Telcagepant Potassium Amorphous Form

Modulated DSC data were acquired using a TA Instruments DSC Q1000. MDSC uses a sinusoidal or modulated change in the heating rate instead of a single linear heating rate, as used in the traditional DSC. This allows the heat flow to be separated into reversing and nonreversing components. The glass transition of amorphous material is detected in the reversing heat flow curve as a change in the baseline, due to a change of the heat capacity of the sample.

Between 2 and 6 mg of sample of telcagepant amorphous potassium salt was weighed into an open pan. This pan was covered with a lid, but not crimped, to allow for any adsorbed moisture to be removed. The pan was placed in the sample position in the calorimeter cell. An empty pan was placed in the reference position. The calorimeter cell was closed and a flow of nitrogen was passed through the cell. The heating program was set to heat the sample at a heating rate of 2° C./min with a modulation period of 60 seconds and modulation amplitude of ±0.5° C. When the run was completed, the data were analyzed using the DSC analysis program in the system software.

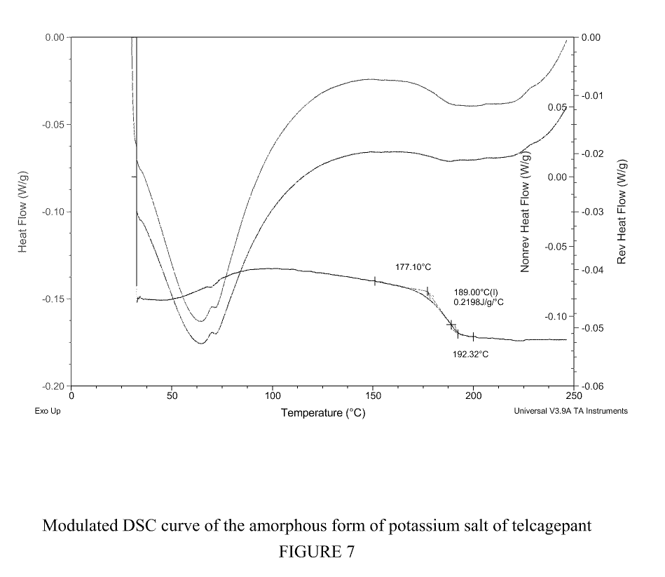

FIG. 7 is a modulated DSC curve of the amorphous potassium salt. The heat capacity change observed in the reversing heat flow curve with a midpoint temperature of 189.00° C. corresponds to the glass transition of amorphous potassium salt.

### Example 5

### Solid State C13 NMR Spectra of Telcagepant Potassium Forms

In addition to the X-ray powder diffraction patterns described above, telcagepant potassium ethanolate was further characterized by solid-state carbon-13 nuclear magnetic resonance (NMR) spectra. The solid-state carbon-13 NMR spectra were obtained on a Bruker DSX 400WB NMR system using a Bruker 4 mm H/X CPMAS probe. The carbon-13 NMR spectra utilized proton/carbon-13 cross-polarization magic-angle spinning with variable-amplitude cross polarization, total sideband suppression, and TPPM decoupling at lOOkHz. The samples were spun at 10.0 kHz, and a total of 512 scans were collected with a recycle delay of 90 seconds. A line broadening of 10 Hz was applied to the spectra before FT was performed. Chemical shifts are reported on the TMS scale using the carbonyl carbon of glycine (176.03 p.p.m.) as a secondary reference.

Form I is characterized by solid-state carbon-13 NMR spectra peaks of 109.1 ppm, 55.8 ppm and 54.6 ppm.

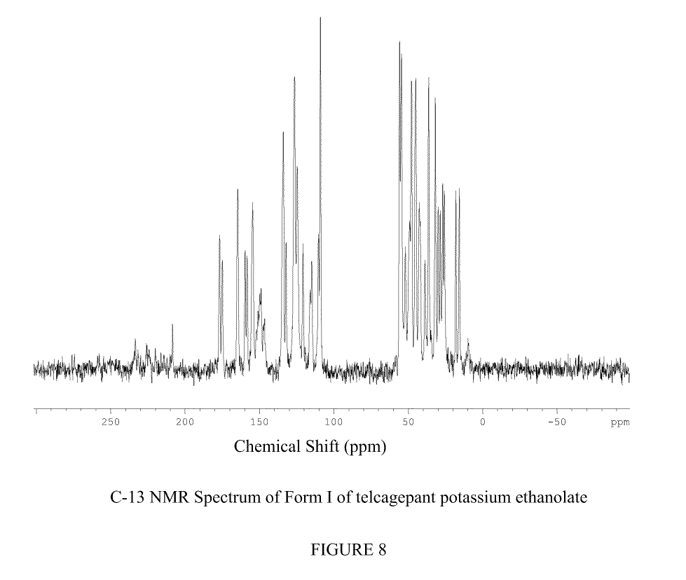

TABLE 2A
Chemical Shift and Relative Intensity for FIG. 8 (Form I telcagepant
potassium ethanolate)

| | Relative |
|---|---|
| Peak (ppm) | Intensity |
| 109.1 | 100 |
| 55.8 | 93 |
| 54.6 | 90 |
| 126.4 | 83 |
| 36.3 | 83 |
| 45.0 | 83 |
| 47.9 | 82 |
| 31.9 | 77 |
| 134.0 | 68 |
| 124.7 | 58 |
| 26.8 | 53 |
| 15.7 | 52 |

The hydrate of the potassium salt of telcagepant is characterized by solid-state carbon-13 NMR spectra peaks of 126.1 ppm, 54.4 ppm and 36.6 ppm.

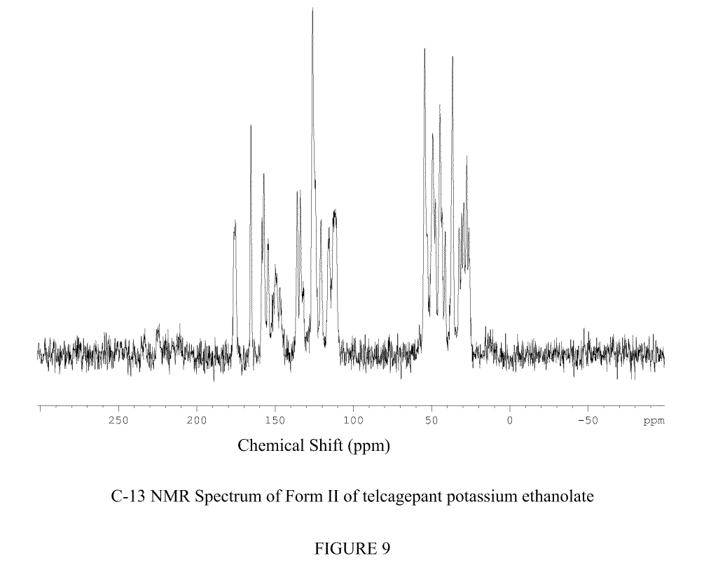

TABLE 2B
Chemical Shift and Relative Intensity for FIG. 9
(telcagepant potassium potassium hydrate)

| Peak | Relative |
|---|---|
| (ppm) | Intensity |
| 126.1 | 100 |
| 54.4 | 88 |
| 36.6 | 86 |
| 44.7 | 72 |
| 165.5 | 66 |
| 49.3 | 64 |
| 27.5 | 57 |
| 157.2 | 52 |
| 133.8 | 47 |
| 135.9 | 47 |
| 47.7 | 45 |
| 29.5 | 44 |
| 111.4 | 42 |
| 30.8 | 41 |
| 158.4 | 39 |
| 175.4 | 39 |
| 120.7 | 39 |
| 32.4 | 37 |
| 115.5 | 36 |
| 26.2 | 36 |
| 41.3 | 35 |
| 154.3 | 33 |

The amorphous form of the potassium salt of telcagepant is characterized by solid-state carbon-13 NMR spectra peaks of 126.0 ppm, 53.7 ppm and 29.1 ppm.

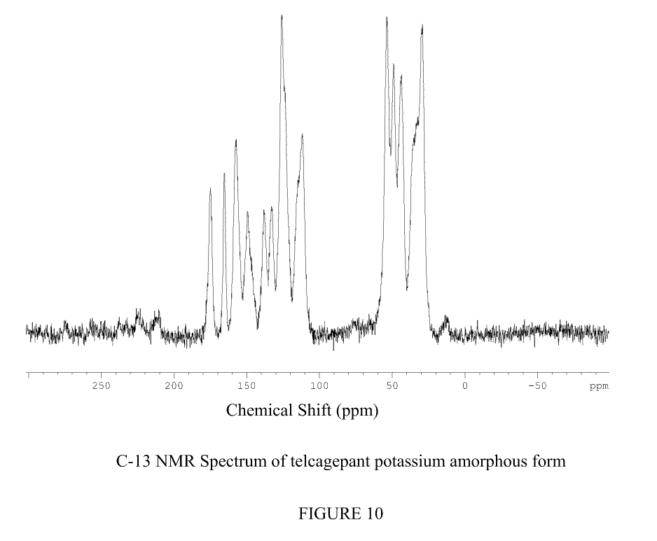

TABLE 2C
Chemical Shift and Relative Intensity for FIG. 10
(amorphous telcagepant potassium)

| Peak | Relative |
|---|---|
| (ppm) | Intensity |
| 126.0 | 100 |
| 53.7 | 99 |
| 29.1 | 97 |
| 49.0 | 85 |
| 43.5 | 81 |
| 111.6 | 63 |
| 157.2 | 61 |
| 165.5 | 50 |
| 174.9 | 46 |
| 132.9 | 40 |
| 138.2 | 39 |
| 149.3 | 38 |

### Example 6

### Raman Spectra of Telcagepant Potassium Forms

Conditions:
Instrumentation: HoloLab Series 5000 by Kaiser Optical Systems, Inc. with an insertion probe.
Sample condition: solid powders without pretreatment.
Sampling mode: each spectrum was collected with 5 seconds of exposure and 5 accumulation

TABLE 3
Main Raman spectral peaks of telcagepant potassium forms:

| Crystal Form | Raman Peaks (cm⁻¹) |
|---|---|
| Ethanolate | 646.3, 707.4, 761.5, 832.9, 1063.3, 1365.5, |
| Form I | 1402.0, 1445.7, 1455.3 |
| Hydrate | 646.8, 707.0, 753.7, 832.7, 1064.7, 1364.3, 1403.0, 1441.0 |
| Amorphous | 646.8, 706.8, 752.3, 832.4, 1063.6, 1365.2, 1437.6 |

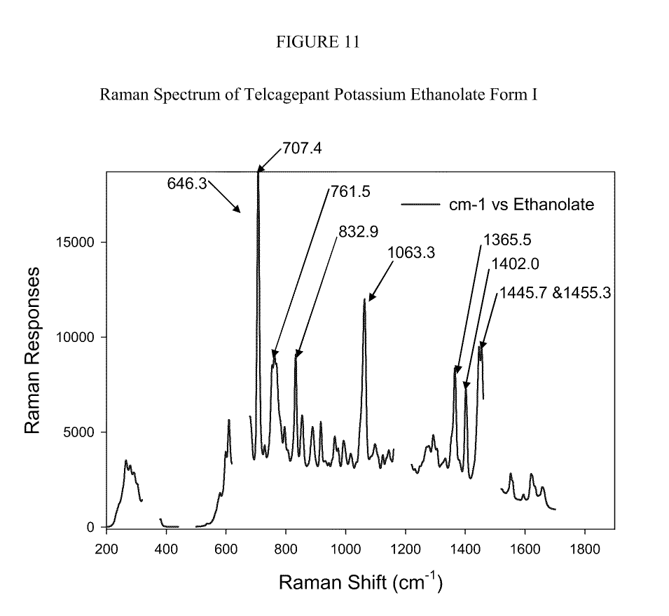

The spectra are depicted in FIGS. 11 (telcagepant potassium ethanolate Form I), 12 (hydrate) and 13 (amorphous form).

### Example 7

### Relative Stability of Telcagepant Potassium Ethanolate Form I and Form II

Slurry experiments were performed at 5° C. and 40° C., adding equal amounts of Form I and Form II to ethanol. The XPRD of the solids recovered from the slurry experiments showed form conversion to Form I, suggesting that Form I is the more stable form in the temperature range of 5° C. and 40° C.

### Example 8

### Exemplary Manufacture of a Tablet

The formulations of the invention may be prepared by a dry granulation method. The tablet manufacturing process is the same for all proposed formulations and drug substance forms. As indicated in the manufacturing process flow diagram shown below, a suitable process according to the invention consists of the following steps:
1. Telcagepant Potassium, Arginine, Mannitol, Poloxamer 407, Silicon Dioxide, and Crospovidone are co-sieved.
2. The sieved material is blended in a suitable blender for about 10 minutes and then lubricated with ½ of batch quantity of Magnesium Stearate.
3. The powder mix is dry granulated using a roller compactor.
4. The resulting compacted granulation is milled.
5. The milled granulation is lubricated with the remaining Magnesium Stearate.
6. The lubricated material is compressed into tablets.
7. The tablets are coated with the white film coating suspension, comprised of Purified Water and OPADRY® White, Brown or other colors.

### Example 9

### Exemplary Formulations of the Potassium Salt of Telcagepant

Exemplary tablet formulations of the potassium salt of telcagepant are shown below in Tables 4A (Form I ethanolate), 4B (hydrate) and 4C (amorphous form).

TABLE 4A
Tablet Formulations of Form I Telcagepant Potassium Ethanolate

| | COMPENDIAL | | UNIT STRENGTH | | |
|---|---|---|---|---|---|
| | TESTING | FUNCTION | 300 mg | 300 mg | 300 mg |
| CORE | | | | | |
| COMPONENTS | | | | | |
| Telcagepant | — | Active | 345.60 | 345.60 | 345.60 |
| Potassium | | Ingredient | | | |
| Ethanolate Form I | | | | | |
| Poloxamer 407 | NF | Surfactant | 60.99 | 34.56 | 34.56 |
| Arginine | USP | Basifying | 243.95 | 172.80 | 103.68 |
| | | Agent | | | |
| Mannitol | USP/NF | Filler | 119.94 | 101.95 | 171.07 |
| Crospovidone | NF | Disintegrant | 28.46 | 24.19 | 24.19 |
| Silicone Dioxide | NF | Glidant | 4.07 | 3.46 | 3.46 |
| Magnesium | NF | Lubricant | 5.08 | 4.32 | 4.32 |
| Stearate | | | | | |
| (intragranular) | | | | | |
| Magnesium | NF | Lubricant | 5.08 | 4.32 | 4.32 |
| Stearate | | | | | |
| (extragranular) | | | | | |
| TOTAL CORE | | | 813.18 | 691.20 | 691.20 |
| WEIGHT | | | | | |
| FILM COAT | | | | | |
| COMPONENTS | | | | | |
| OPADRY White | — | Film Coat | 24.40 | 20.74 | 20.74 |
| Film Coat Blend | | | | | |

| | | | | | |
|---|---|---|---|---|---|
| WATER | USP | Solvent | N/A | N/A | N/A |

| | | | | | | |
|---|---|---|---|---|---|---|
| THEORETICAL | 838 | mg | 712 | mg | 712 | mg |
| COATED | | | | | | |
| WEIGHT | | | | | | |

TABLE 4B
Tablet Formulation of Hydrate of Potassium Salt

| | | | UNIT |
|---|---|---|---|
| | COMPENDIAL | | STRENGTH |
| | TESTING | FUNCTION | 300 mg |
| CORE | | | |
| COMPONENTS | | | |
| Telcagepant | — | Active | 347.1 |
| Potassium Hydrate | | Ingredient | |
| Poloxamer 407 | NF | Sufactant | 34.71 |
| Arginine | USP | Basifying Agent | 173.55 |
| Mannitol | USP/NF | Filler | 102.39 |
| Crospovidone | NF | Disintegrant | 24.30 |
| Silicone Dioxide | NF | Glidant | 3.47 |
| Magnesium | NF | Lubricant | 4.34 |
| Stearate | | | |
| (intragranular) | | | |
| Magnesium | NF | Lubricant | 4.34 |
| Stearate | | | |
| (extragranular) | | | |
| TOTAL CORE | | | 694.20 |
| WEIGHT | | | |
| FILM COAT | | | |
| COMPONENTS | | | |
| OPADRY I White | | Film Coat | 20.83 |
| Film Coat Blend | | | |

| | | | |
|---|---|---|---|
| WATER | USP | Solvent | N/A |

| | | |
|---|---|---|
| THEORETICAL | 715 | mg |
| COATED | | |
| WEIGHT | | |

TABLE 4C
Tablet Formulations of Amorphous Form of Potassium Salt

| | | | UNIT |
|---|---|---|---|
| | COMPENDIAL | | STRENGTH |
| | TESTING | FUNCTION | 300 mg |
| CORE | | | |
| COMPONENTS | | | |
| Telcagepant | — | Active | 320.10 |
| Potassium | | Ingredient | |
| Amorphous Form | | | |
| Poloxamer 407 | NF | Sufactant | 32.01 |
| Arginine | USP | Basifying Agent | 160.05 |
| Mannitol | USP/NF | Filler | 94.43 |
| Crospovidone | NF | Disintegrant | 22.41 |
| Silicone Dioxide | NF | Glidant | 3.20 |
| Magnesium | NF | Lubricant | 4.00 |
| Stearate | | | |
| (intragranular) | | | |
| Magnesium | NF | Lubricant | 4.00 |
| Stearate | | | |
| (extragranular) | | | |
| TOTAL CORE | | | 640.20 |
| WEIGHT | | | |
| FILM COAT | | | |
| COMPONENTS | | | |
| OPADRY White | | Film Coat | 19.21 |
| Film Coat Blend | | | |
| WATER | Solvent | | N/A |
| THEORETICAL | | | 659 |
| COATED | | | |
| WEIGHT | | | |

### Example 10

### Comparative Study of Formulations of Telcagepant

An open-label, randomized, 6-period crossover study was conducted to determine the comparative bioavailability of six formulations of telcagepant, administered as single oral doses to 36 healthy male and female subjects. The six formulations included five solid dosage formulations (Table 5), and an oral soft elastic liquid filled capsule (C1). Three of the solid dosage forms contained Form I telcagepant potassium, another contained the telcagepant potassium hydrate, and the fifth contained the amorphous form of telcagepant potassium.

The formulations are described below in Table 5.

TABLE 5
Solid Dosage Formulations by Weight Percent of Potassium
Salt of Telcagepant

| Ingredient | F | G | H | I | J |
|---|---|---|---|---|---|
| Ethanolate of | 42.50% | 50.00% | 50.00% | | |
| Potassium Salt | | | | | |
| of Telcagepant | | | | | |
| (Form I) | | | | | |
| Hydrate of | | | | 50.00% | |
| Potassium Salt | | | | | |
| of Telcagepant | | | | | |
| Amorphous | | | | | 50.00% |
| Form of | | | | | |
| Potassium Salt | | | | | |
| of Telcagepant | | | | | |
| Poloxamer 407 | 7.50% | 5.00% | 5.00% | 5.00% | 5.00% |
| Arginine | 30.00% | 25.00% | 15.00% | 25.00% | 25.00% |
| Other | 20.00% | 20.00% | 30.00% | 20.00% | 20.00% |
| Excipients | | | | | |

The “other excipients” included in the formulations were magnesium stearate, crospovidone, silicone dioxide, mannitol and coating.

Also included in the study was a liquid filled oral soft elastic capsule formulation, C1, which comprised the following ingredients:

| | |
|---|---|
| Telcagepant potassium salt ethanolate | 28.56% |
| PEG 400 | 23.36% |
| Propylene Glycol | 7.14% |
| Cremophor EL | 18.09% |
| Polysorbate 80 | 18.09% |
| Butylated hydroxyl toluene | 0.04% |
| Water | 4.72% |

After an overnight 8-hour fast, each subject received a single 300-mg oral dose of 1 of the 6 formulations, administered with 240 mL of water. Water was restricted 1 hour prior to and after drug administration and the order in which the subjects received each dose was randomized according to a computer generated allocation schedule. Each treatment period was separated by a minimum washout of 5 days.

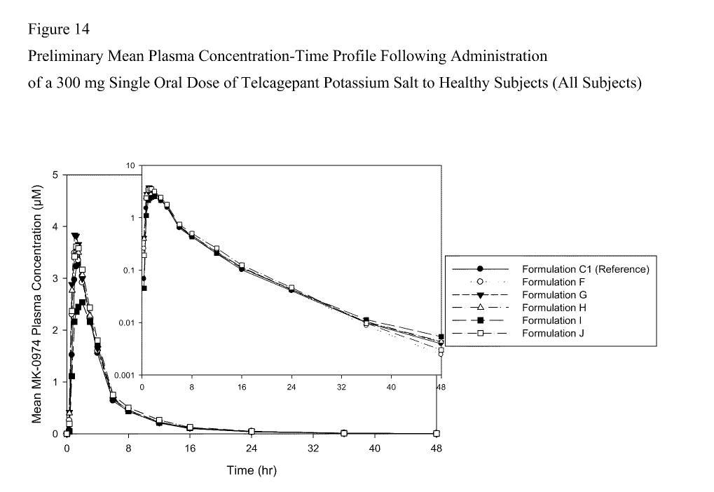

The shape of the mean plasma concentration-time profile following administration of a single dose of the telcagepant formulation was not appreciably different from that for Formulation C₁, the oral liquid filled capsule (FIG. 14). Profiles from each formulation suggest rapid absorption (median Tₘₐₓ≦1.5 hr), with similar Tₘₐₓ across formulations and at least a bi-exponential decline in telcagepant plasma concentration post-peak with a similar apparent terminal half-life across formulations (FIG. 14).

Tables 6A-6G below presents the results of the statistical analysis of various pharmacokinetic data from the study. The following definitions are relevant:

GM=geometric mean

GMR vs. C1=geometric mean ratio vs. C1

HM=harmonic mean

% CV=% coefficient of variation

90% C1=90% confidence interval

AUC=The “AUC,” or “Area under the Curve,” is a measure of the plasma concentration of the drug over time, and is a measure of drug exposure. Measurement of AUC is well known to those skilled in the art of formulation.

Cmax=Cmax is a measure of the highest plasma drug concentration observed.

Tmax=Tmax is the time when Cmax is first reached

Half-life=The period of time required for the concentration or amount of drug in the body to be reduced by one-half.

Further explanations of these terms can be found in Goodman and Gilman's The Pharmacological Basis of Therapeutics, pp. 18-19, ₁₇₉₀-₁₇₉₁ (11^{th} ed. 2006).

### Tables 6A-G —Summary of Pharmacokinetic Results Following Single-Dose Administration of Six Formulations of Telcagepant

TABLE 6A
Measure of AUC_{0-∞} (μM hr)

| | Formulation | | | | | |
|---|---|---|---|---|---|---|
| Value | C₁ | F | G | H | I | J |
| GM | 13.15 (49) | 10.79 (369) | 14.49 (45) | 14.96 (40) | 11.09 (131) | 11.57 (380) |
| (% CV) | | | | | | |
| GMR | N/A | 0.82 | 1.10 | 1.14 | 0.84 | 0.88 |
| vs. C₁ | | | | | | |

TABLE 6B
Cₘₐₓ (μM)

| | Formulation | | | | | |
|---|---|---|---|---|---|---|
| Value | C₁ | F | G | H | I | J |
| GM | 3.56 | 3.13 | 4.14 | 4.07 | 2.87 (124) | 3.18 |
| (% CV) | (46) | (248) | (45) | (40) | | (249) |
| GMR | | 0.88 | 1.16 | 1.14 | 0.81 | 0.89 |
| vs. C₁ | | | | | | |

TABLE 6C
AUC_{0-4 hr} (μM hr)

| | Formulation | | | | | |
|---|---|---|---|---|---|---|
| Value | C₁ | F | G | H | I | J |
| GM (% | 7.89 (48) | 6.72 (312) | 8.89 (46) | 8.90 (41) | 6.07 (126) | 7.07 (319) |
| CV) | | | | | | |
| GMR | | 0.85 | 1.13 | 1.13 | 0.77 | 0.90 |
| vs. C₁ | | | | | | |
| 90% CI | | (0.61, 1.19) | (0.81, 1.57) | (0.81, 1.57) | (0.55, 1.07) | (0.64, 1.25) |

TABLE 6D
AUC_{0-2 hr} (μM hr)

| | Formulation | | | | | |
|---|---|---|---|---|---|---|
| Value | C₁ | F | G | H | I | J |
| GM | 3.50 (76) | 3.39 (272) | 4.25 (102) | 4.03 (111) | 2.13 (182) | 3.50 (272) |
| (% CV) | | | | | | |
| GMR | | 0.97 | 1.22 | 1.15 | 0.61 | 1.00 |
| vs. C₁ | | | | | | |
| 90% CI | | (0.67, 1.41) | (0.84, 1.76) | (0.79, 1.67) | (0.42, 0.89) | (0.69, 1.45) |

TABLE 6E
AUC_{0-Tmax} (μM hr)

| | Formulation | | | | | |
|---|---|---|---|---|---|---|
| Value | C₁ | F | G | H | I | J |
| GM (% | 2.02 (63) | 2.25 (216) | 2.35 (64) | 2.47 (65) | 1.81 (167) | 2.49 (254) |
| CV) | | | | | | |
| GMR | | 1.12 | 1.16 | 1.22 | 0.89 | 1.23 |
| vs. C₁ | | | | | | |
| 90% CI | | (0.88, 1.42) | (0.92, 1.47) | (0.96, 1.55) | (0.71, 1.13) | (0.97, 1.57) |

TABLE 6F
Tₘₐₓ (hr)

| | Formulation | | | | | |
|---|---|---|---|---|---|---|
| Value | C₁ | F | G | H | I | J |
| Median | 1.38 | 1.25 | 1.25 | 1.25 | 1.50 | 1.50 |
| Min, | 1.00, | 1.00, | 0.67, | 0.67, | 1.00, 4.00 | 0.67, |
| Max | 3.00 | 3.00 | 4.00 | 4.00 | | 4.00 |
| Med. | | −0.13 | −0.13 | 0.00 | 0.38 | 0 |
| Diff vs. | | | | | | |
| C₁ | | | | | | |
| 90% CI | | (−0.25, | (−0.25, | (−0.29, | (0.00, 0.75) | (−0.25, |
| | | 0.13) | 0.13) | 0.25) | | 0.25) |

TABLE 6G
Half-life (hr)

| | Formulation | | | | | |
|---|---|---|---|---|---|---|
| Value | C₁ | F | G | H | I | J |
| HM | 5.5 | 5.3 | 5.8 (2.2) | 5.7 (2.5) | 6.0 (2.8) | 5.5 (1.9) |
| (Pseudo | (2.2) | (1.4) | | | | |
| SD) | | | | | | |

There were no statistically significant differences in Tₘₐₓ between the test formulations and the reference liquid filled capsule.

### Example 11

### Comparison of Formulation of Telcagepant Potassium Ethanolate and Telcagepant Potassium Hydrate

Administration of telcagepant formulation C1 (described above), liquid filled capsule (300 and 600 mg) resulted in 2-hour pain freedom and pain relief counts that were superior to placebo in a Phase II study. Administration of telcagepant formulation C1 (150 mg and 300 mg) resulted in 2-hour pain freedom and pain relief counts that were superior to placebo in a Phase III study.

A solid formulation of the ethanolate salt of telcagepant, formulation G1, was compared to C1 in this study. This study directly compared the pharmacokinetic profiles of 280 mg telcagepant ethanolate salt (Formulation G1 tablet, a slightly modified Formulation G tablet) to 280 mg telcagepant hydrate (Formulation I tablet) in a randomized, cross-over fashion.

TABLE 7
Weight percent Descriptions of Formulations G1 and I

| Ingredients | G1 | I |
|---|---|---|
| Ethanolate | 50.00% | |
| of Potassium | | |
| Salt of | | |
| Telcagepant | | |
| (Form I) | | |
| Hydrate of | | 50.00% |
| Potassium | | |
| Salt of | | |
| Telcagepant | | |
| Poloxamer | 5.00% | 5.00% |
| 407 | | |
| Arginine | 25.00% | 25.00% |
| Other | 20.00% | 20.00% |
| Excipients | | |

An open-label, randomized, 2-period crossover study was conducted to evaluate the bioequivalence of two formulations (formulations G1 and I) of telcagepant administered as single oral doses to 36 healthy male and female subjects.

Each subject received each dose of telcagepant at the same time in both periods. After an overnight 8-hour fast, each subject received either a single 280-mg oral dose of solid dose Formulation G1 or a single 280-mg oral dose of solid dose Formulation I. These doses were administered with 240 mL of water. Water was restricted 1 hour prior to and after drug administration and the order in which the subjects receive each dose was randomized according to a computer generated allocation schedule. Subjects had blood collected at predose and at specified time points over 48 hours following drug administration in both periods for pharmacokinetic measurements. Subjects were sequestered at the clinical research unit (CRU) for 24 hours post dose in both treatment periods for pharmacokinetic measurements. Subjects may have been required to remain in the research unit up to 48 hours post-dose, at the discretion of the investigator. There was a minimum washout of 5 days (˜15 half-lives), between the treatment periods. Safety and tolerability was assessed by careful questioning for adverse events, ECGs, monitoring of vital signs, and laboratory safety assessments.

### Results

The shape of the mean plasma concentration-time profile of the two formulations of telcagepant was not appreciably different, with both profiles suggesting rapid absorption and at least a bi-exponential decline in telcagepant plasma concentration post-peak.

Table 8 presents the results of the statistical analysis of the pharmacokinetic data. For the comparison of 280-mg solid dose Formulation G1 to 280-mg telcagepant solid dose hydrate formulation, the geometric mean ratio (Formulation G1/Formulation I) and corresponding 90% confidence interval for AUC0-∞ and Cmax were 0.94 (0.88, 0.99) and 0.95 (0.83, 1.08), respectively.

The following definitions are relevant:

GM=geometric mean

MSE=mean square error

% CV=% coefficient of variation

90% CI=90% confidence interval

AUC=The “AUC,” or “Area under the Curve,” is a measure of the plasma concentration of the drug over time, and is a measure of drug exposure. Measurement of AUC is well known to those skilled in the art of formulation.

Cmax=Cmax is a measure of the highest plasma drug concentration observed.

Tmax=Tmax is the time when Cmax is first reached

Half-life=The period of time required for the concentration or amount of drug in the body to be reduced by one-half.

TABLE 8
Summary of Pharmacokinetic Results Following Single-Dose
Administration of 280 mg of Telcagepant Solid Dose
Form I Ethanolate (G1) Formulation and
Hydrate Formulation (I) to Healthy Subjects

| | GM for Treatment (% CV) | | | |
|---|---|---|---|---|
| | 280 mg | | GM (90% CI) | |
| Pharmaco- | Ethanolate | 280 mg | for Treatment | |
| kinetic | (Formulation | Hydrate | Ratio | |
| Parameter | G₁) | (Formulation I) | (G₁/I) | MSE |
| AUC_{0-∞} | 14.28 (42) | 15.25 (43) | 0.94 (0.88, 0.99) | 0.0235 |
| (μM · hr) | | | | |
| Cₘₐₓ (μM) | 4.55 (54) | 4.80 (41) | 0.95 (0.83, 1.08) | 0.110 |
| AUC₀₋₄ | 9.22 (43) | 9.87 (42) | 0.93 (0.86, 1.01) | 0.0382 |
| (μM · hr) | | | | |
| AUC₀₋₂ | 4.70 (60) | 5.46 (47) | 0.86 (0.75, 0.99) | 0.118 |
| (μM · hr) | | | | |
| AUC_{0-Tmax} | 1.41 (99) | 1.64 (85) | 0.86 (0.65, 1.12) | 0.462 |
| (μM · hr) | | | | |
| Tₘₐₓ (hr) | 1.00 [0.67, | 1.00 [0.67, | 0.085 (−0.13, | |
| | 3.00] | 3.00] | 0.34) | |
| Half-life (hr) | 6.5 (2.0) | 6.2 (1.8) | | |

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, effective dosages other than the particular dosages as set forth herein above may be applicable as a consequence of variations in the responsiveness of the mammal being treated for any of the indications with the compounds of the invention indicated above. Likewise, the specific pharmacological responses observed may vary according to and depending upon the particular active compounds selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

What is claimed is:

1. A solid dosage pharmaceutical formulation comprising
(1) N-[(3R,6S)-6-(2,3-difluorophenyl)-2-oxo-1-(2,2,2-trifluoroethyl)azepan-3-yl]-4-(2-oxo-2,3-dihydro-1H-imidazo[4,5-b]pyridin-1-yl)piperidine-1-carboxamide potassium, or the hydrate or ethanolate thereof, or an amorphous form thereof,
(2) arginine; and
(3) a pharmaceutically acceptable surfactant.

2. The solid dosage pharmaceutical formulation of claim 1, wherein arginine is present in the amount of at least about 10% by weight of the formulation.

3. The solid dosage pharmaceutical formulation of claim 1 or 2, comprising about 100 to about 500 mg of N-[(3R,6S)-6-(2,3-difluorophenyl)-2-oxo-1-(2,2,2-trifluoroethyl)azepan-3-yl]-4-(2-oxo-2,3-dihydro-1H-imidazo[4,5-b]pyridin-1-yl)piperidine-1-carboxamide potassium.

4. The solid dosage pharmaceutical formulation of any of claims 1 to 3, comprising about 35 to about 55% by weight of N-[(3R,6S)-6-(2,3-difluorophenyl)-2-oxo-1-(2,2,2-trifluoroethyl)azepan-3-yl]-4-(2-oxo-2,3-dihydro-1H-imidazo[4,5-b]pyridin-1-yl)piperidine-1-carboxamide potassium.

5. The solid dosage pharmaceutical formulation of any of claims 1 to 4,
wherein the pharmaceutically acceptable surfactant is a nonionic surfactant.

6. The solid dosage pharmaceutical formulation of claim 5 wherein the nonioinic surfactant is a polyoxypropylene block copolymer.

7. The solid dosage pharmaceutical formulation of any of claims 1 to 6
wherein the surfactant is present in the amount of up to about 10% by weight of the formulation.

8. The solid dosage pharmaceutical formulation of any of claims 1 to 7, which comprises N-[(3R,6S)-6-(2,3-difluorophenyl)-2-oxo-1-(2,2,2-trifluoroethyl)azepan-3-yl]-4-(2-oxo-2,3-dihydro-1H-imidazo[4,5-b]pyridin-1-yl)piperidine-1-carboxamide potassium ethanolate Form I.

9. The solid dosage pharmaceutical formulation of any of claims 1 to 7, which comprises N-[(3R,6S)-6-(2,3-difluorophenyl)-2-oxo-1-(2,2,2-trifluoroethyl)azepan-3-yl]-4-(2-oxo-2,3-dihydro-1H-imidazo[4,5-b]pyridin-1-yl)piperidine-1-carboxamide potassium ethanolate Form II.

10. The solid dosage pharmaceutical formulation of any of claims 1 to 7, which comprises N-[(3R,6S)-6-(2,3-difluorophenyl)-2-oxo-1-(2,2,2-trifluoroethyl)azepan-3-yl]-4-(2-oxo-2,3-dihydro-1H-imidazo[4,5-b]pyridin-1-yl)piperidine-1-carboxamide potassium hydrate.

11. The solid dosage pharmaceutical formulation of any of claims 1 to 7, which comprises amorphous N-[(3R,6S)-6-(2,3-difluorophenyl)-2-oxo-1-(2,2,2-trifluoroethyl)azepan-3-yl]-4-(2-oxo-2,3-dihydro-1H-imidazo[4,5-b]pyridin-1-yl)piperidine-1-carboxamide potassium.

12. The solid dosage pharmaceutical formulation of claim 8, wherein the N-[(3R,6S)-6-(2,3-difluorophenyl)-2-oxo-1-(2,2,2-trifluoroethyl)azepan-3-yl]-4-(2-oxo-2,3-dihydro-1H-imidazo[4,5-b]pyridin-1-yl)piperidine-1-carboxamide potassium ethanolate Form I displays solid-state carbon-13 NMR spectra peaks of one or more of 109.1 ppm, 55.8 ppm and 54.6 ppm.

13. The solid dosage pharmaceutical formulation of claim 8, wherein the Raman spectra of N-[(3R,6S)-6-(2,3-difluorophenyl)-2-oxo-1-(2,2,2-trifluoroethyl)azepan-3-yl]-4-(2-oxo-2,3-dihydro-1H-imidazo[4,5-b]pyridin-1-yl)piperidine-1-carboxamide potassium ethanolate Form I displays peaks (cm⁻¹) of one or more of 646.3, 707.4, 761.5, 832.9, 1063.3, 1365.5, 1402.0, 1445.7 and 1455.3

14. The solid dosage pharmaceutical formulation of claim 10, wherein the N-[(3R,6S)-6-(2,3-difluorophenyl)-2-oxo-1-(2,2,2-trifluoroethyl)azepan-3-yl]-2-(2-oxo-2,3-dihydro-1H-imidazo[4,5-b]pyridin-1-yl)piperidine-1-carboxamide potassium hydrate displays solid-state carbon-13 NMR spectra peaks of one or more of 126.1 ppm, 54.4 ppm and 36.6 ppm.

15. The solid dosage pharmaceutical formulation of claim 10, wherein the Raman spectra of N-[(3R,6S)-6-(2,3-difluorophenyl)-2-oxo-1-(2,2,2-trifluoroethyl)azepan-3-yl]-4-(2-oxo-2,3-dihydro-1H-imidazo[4,5-b]pyridin-1-yl)piperidine-1-carboxamide potassium hydrate displays peaks (cm⁻¹) of one or more of 646.8, 707.0, 753.7, 832.7, 1064.7, 1364.3, 1403.0 and 1441.0

16. The solid dosage pharmaceutical formulation of claim 11, wherein the N-[(3R,6S)-6-(2,3-difluorophenyl)-2-oxo-1-(2,2,2-trifluoroethyl)azepan-3-yl]-4-(2-oxo-2,3-dihydro-1H-imidazo[4,5-b]pyridin-1-yl)piperidine-1-carboxamide potassium amorphous form displays solid-state carbon-13 NMR spectra peaks of one or more of 126.0 ppm, 53.7 ppm and 29.1 ppm.

17. The solid dosage pharmaceutical formulation of claim 11, wherein the Raman spectra of the N-[(3R,6S)-6-(2,3-difluorophenyl)-2-oxo-1-(2,2,2-trifluoroethyl)azepan-3-yl]-4-(2-oxo-2,3-dihydro-1H-imidazo[4,5-b]pyridin-1-yl)piperidine-1-carboxamide potassium amorphous form displays peaks (cm⁻¹) of one or more of 646.8, 706.8, 752.3, 832.4, 1063.6, 1365.2 and 1437.6.

18. The solid dosage pharmaceutical formulation of any of claims 1 to 17, which comprises about 280 mg or about 300 mg of the active ingredient N-[(3R,6S)-6-(2,3-difluorophenyl)-2-oxo-1-(2,2,2-trifluoroethyl)azepan-3-yl]-4-(2-oxo-2,3-dihydro-1H-imidazo[4,5-b]pyridin-1-yl)piperidine-1-carboxamide.

19. The solid dosage pharmaceutical formulation of any of claims 1 to 18, which is a tablet.

20. The solid dosage pharmaceutical formulation of any of claims 1 to 19, which provides Cmax in the blood of at least 2.75 μM.

21. The solid dosage pharmaceutical formulation of any of claims 1 to 19,
which achieves a Tmax at a time point of no more than 1.0 hour after administration.

22. The solid dosage pharmaceutical formulation of any of claims 1 to 19,
which achieves an AUC_{0-Tmax} in the blood of no more than 2.5 μM.

23. The solid dosage pharmaceutical formulation of any of claims 1 to 19,
which achieves an AUC_{0-2 hr} in the blood of no more than 5.5 μM.

24. The solid dosage pharmaceutical formulation of any of claims 1 to 19,
which achieves an AUC₀₋₄ hr in the blood of no more than 10.0 μM.

25. The solid dosage pharmaceutical formulation of any of claims 1 to 19,
which achieves an AUC_{0-∞} in the blood of no more than 15.5 μM.

26. An amorphous form of N-[(3R,6S)-6-(2,3-difluorophenyl)-2-oxo-1-(2,2,2-trifluoroethyl)azepan-3-yl]-4-(2-oxo-2,3-dihydro-1H-imidazo[4,5-b]pyridin-1-yl)piperidine-1-carboxamide potassium, said amorphous form produced by the step of spray drying of the ethanolate or hydrate of N-[(3R,6S)-6-(2,3-difluorophenyl)-2-oxo-1-(2,2,2-trifluoroethyl)azepan-3-yl]-4-(2-oxo-2,3-dihydro-1H-imidazo[4,5-b]pyridin-1-yl)piperidine-1-carboxamide potassium in an organic solution.

27. The amorphous form of N-[(3R,6S)-6-(2,3-difluorophenyl)-2-oxo-1-(2,2,2-trifluoroethyl)azepan-3-yl]-4-(2-oxo-2,3-dihydro-1H-imidazo[4,5-b]pyridin-1-yl)piperidine-1-carboxamide potassium of claim 26, said amorphous form having a mean particle size of less than 15 μm.

28. An amorphous form of N-[(3R,6S)-6-(2,3-difluorophenyl)-2-oxo-1-(2,2,2-trifluoroethyl)azepan-3-yl]-4-(2-oxo-2,3-dihydro-1H-imidazo[4,5-b]pyridin-1-yl)piperidine-1-carboxamide potassium, said amorphous form produced by the step of dissolving N-[(3R,6S)-6-(2,3-difluorophenyl)-2-oxo-1-(2,2,2-trifluoroethyl)azepan-3-yl]-4-(2-oxo-2,3-dihydro-1H-imidazo[4,5-b]pyridin-1-yl)piperidine-1-carboxamide potassium ethanolate or hydrate in methanol, and precipitating the amorphous form.

29. The amorphous form of N-[(3R,6S)-6-(2,3-difluorophenyl)-2-oxo-1-(2,2,2-trifluoroethyl)azepan-3-yl]-4-(2-oxo-2,3-dihydro-1H-imidazo[4,5-b]pyridin-1-yl)piperidine-1-carboxamide potassium of claim 28, said amorphous form having a mean particle size of less than 150 μm.
